SEQUENCE LISTING (22 sequences)

SEQ ID NO: 1 - protein, 10 aa, synthetic construct
LLLLLCCCCC

SEQ ID NO: 2 - protein, 10 aa, synthetic construct
  SITE 6..10: note = S-benzyl-L-cysteine
LLLLLXXXXX

SEQ ID NO: 3 - protein, 20 aa, synthetic construct
  SITE 16..20: note = S-benzyl-L-cysteine
LLLLLLLLLLLLLLLXXXXX

SEQ ID NO: 4 - protein, 19 aa, synthetic construct
  SITE 11..19: note = S-benzyl-L-cysteine
LLLLLLLLLLXXXXXXXXX

SEQ ID NO: 5 - protein, 15 aa, synthetic construct
  SITE 11..15: note = S-benzyl-L-cysteine
LLLLLLLLLLXXXXX

SEQ ID NO: 6 - protein, 10 aa, synthetic construct
  SITE 6..10: note = S-benzyl-L-cysteine
LLLLLXXXXX

SEQ ID NO: 7 - protein, 15 aa, synthetic construct
LLLLLLLLLLLLLLL

SEQ ID NO: 8-12 - skipped (5 entries)

SEQ ID NO: 13 - protein, 10 aa, synthetic construct
LLLLLLLLLL

SEQ ID NO: 14 - skipped

SEQ ID NO: 15 - protein, 9 aa, synthetic construct
LLLLLLLLL

SEQ ID NO: 16 - skipped

SEQ ID NO: 17 - protein, 15 aa, synthetic construct
LLLLLLLLLLFFFFF

SEQ ID NO: 18 - skipped

SEQ ID NO: 19 - protein, 15 aa, synthetic construct
LLLLLLLLLLCCCCC

SEQ ID NO: 20 - protein, 20 aa, synthetic construct
LLLLLLLLLLLLLLLCCCCC

SEQ ID NO: 21 - protein, 25 aa, synthetic construct
  SITE 21..25: note = S-Benzyl-L-cysteine
LLLLLLLLLLLLLLLLLLLLXXXXX

SEQ ID NO: 22 - protein, 25 aa, synthetic construct
LLLLLLLLLLLLLLLLLLLLCCCCC

(19) United States
(12) Patent Application Publication
(10) Pub. No.: US 2024/0228537 A1
(43) Pub. Date: Jul. 11, 2024

(54) BLOCK COPOLYPEPTIDES AND USE THEREOF

(71) Applicant: National Cheng Kung University, Tainan City (TW)

(72) Inventors: JENG-SHIUNG JAN, Tainan City (TW); YU-FON CHEN, Tainan City (TW)

(21) Appl. No.: 18/150,733

(22) Filed: Jan. 5, 2023

(51) Int. Cl. C07K 7/00; A61K 45/06; A61P 35/00

(52) U.S. Cl. CPC C07K 7/00; A61K 45/06; A61P 35/00

(57) ABSTRACT

Provided is a block copolypeptide comprising a first positively charged peptide segment consisting of 5 to 20 substituted or unsubstituted L-lysine, L-arginine, L-ornithine, or L-homoarginine. The block copolypeptide can suppresse the migration and adhesion of cancer cells, reduce tumor growth, and modulate intestinal microbiota, particularly increasing the population of Akkermansia muciniphila. Provided are also a method for treating cancer and a method for increasing the proportion of Akkermansia muciniphila (Akk) in the gut, including administering an effective amount of the block copolypeptide to the subject in need thereof.

### BACKGROUND OF THE INVENTION

### Sequence Listing

Pursuant to 37 CFR §§ 1.831-835, the instant application contains a computer readable Sequence Listing which has been submitted electronically in XML format and is hereby incorporated by reference in its entirety. Said XML format file, created on Aug. 22, 2023, is named 211142US sequence listing.xml and is 26.3 kb in size.

### TECHNICAL FIELD

The present disclosure relates to block copolypeptides, particularly relates to block copolypeptides comprising a first positively charged peptide segment and a second hydrophobic peptide segment that can be used for suppressing migration and adhesion of cancer cells, reducing tumor growth, treating cancer and modulating intestinal microbiota. The block copolypeptides also serve as gene vectors for improvement of transfection efficiency, endosomal escape, and biocompatibility.

### DESCRIPTION OF THE RELATED ART

Peptide drugs are intermediates between small molecules and protein drugs, with unique biochemical and therapeutic properties that make them distinct from both other extremes and sometimes put them at an advantage in comparison to other drugs. Peptide drugs are less toxic, more specific toward targets than most chemical drugs, and more easily synthesized than protein drugs. Even so, the development of peptide drugs has had a significant impact on clinical medicine over the past two decades; there have been more than 80 peptide drugs approved and hundreds more in preclinical or clinical trials worldwide. However, peptides suffer from a short in vivo half-life, sometimes mere minutes, making them generally impractical, in their native form, for therapeutic administration. Thus, there exists a need in the art for modified therapeutic peptides having an enhanced half-life and/or reduced clearance as well as additional therapeutic advantages as compared to the therapeutic peptides in their unmodified form.

In addition, gene therapy is a curative treatment for patients with genetic-related defects, and the success of such treatments is directly tied to the efficiency of gene vectors. Two distinct types of gene vectors, viral vectors and non-viral vectors are currently used. Viral vectors may present adverse immune responses or sequelae and are considered at higher risk. Non-viral vectors, such as cationic polymers (including polypeptides), relatively have fewer side effects, but generally have poor transfection efficiency. It has been reported that modification of cationic polymers to increase amphiphilicity, including facile side-chain modifications, can improve transfection efficiency.

### SUMMARY OF THE INVENTION

In view of the above-described drawbacks, the present disclosure is based on the discovery that the potentiality of block copolypeptides comprising a first positively charged peptide segment and a second hydrophobic peptide segment on inhibiting migration and adhesion of cancer cells by perturbing integrity of cancer cell membrane and interfering E-cadherin packing on the cancer cell membrane. Also, the present disclosure finds out the block copolypeptides comprising a first positively charged peptide segment and a second hydrophobic peptide segment as gene vectors have improved transfection efficiency, endosomal escape, and biocompatibility.

The processes of cancer metastasis are primarily divided into 4 steps: i) detachment from primary tumors, ii) migration into vascular or lymphatic circulation, iii) adhesion and acclimatization to a secondary site, and iv) transition/growth into new metastatic tumors. E-cadherin plays a critical role in invasive ductal carcinomas during detachment, systemic dissemination, and metastatic phases.

Many metastases present high levels of E-cadherin expression, since epithelial cells expressing E-cadherin can become more invasive and metastasize without involvement of other transition molecules. Of note, E-cadherin conformations are only stable upon Ca²⁺ binding to its highly conserved, negatively charged extracellular motifs. Amino acid derivatives with cationic and bulky functional groups are to inhibit the formation of an intermediate in dimerization of P-cadherin. The cationic group may interact with the negatively charged region around the cavity located between EC1 and EC2 domains of P-cadherin, whilst the bulky functional group such as indole and benzyl groups may protrude the interface to block dimerization. Therefore, designing cell adhesion inhibiting peptides by interfering E-cadherin packaging via the similar strategy is the starting point for the development of a short peptide.

Basically, cancer cell membranes exhibit more anionic components than normal ones, and this property is used to develop cationic and hydrophobic peptides as anticancer agents. In addition, structures that affect cell membrane integrity or protein-protein interaction, such as α-helix, β-sheet and random coil have been incorporated in the design of anti-cancer peptides (ACPs).

As such, in the first aspect, the present disclosure provides a block copolypeptide comprising a first positively charged peptide segment and a second hydrophobic peptide segment for cancer therapy.

Amino acids which constitute a positively charged segment or hydrophobic segment have no particular limitation, and may all be natural amino acids or all be non-natural amino acids, or may comprise natural amino acids and non-natural amino acids at the same time. Similarly, the sequence of amino acids in each segment has no particular limitation, only if the segment constituted by amino acids exhibits the desired property, i.e. having positive charge or hydrophobicity. In addition, the sequence of segments also has no particular limitation.

In at least one embodiment of the present disclosure, the first positively charged peptide segment consists of L-lysine, L-arginine, L-onithine, or L-homoarginine and the second hydrophobic peptide segment consists of L-phenylalanine, L-tryptophan, L-dopa, L-tyrosine, L-cysteine, S-benzyl-L-cysteine, or S-methyl cysteine, wherein the first peptide segment and second peptide segment comprise 5 to 20 amino acids, respectively.

Each of the first peptide segment and second peptide segment may comprise 5, 6, 7, 8, 9, 10, 11, 12, 13, 14, 15, 16, 17, 18, 19, or 20 amino acids. The total length of the block copolypeptide of the present disclosure thus may be 10 to 40 amino acids, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

The first positively charged peptide segment may be consisting of only L-lysine, only L-arginine, only L-ornithine, only L-homoarginine, or two or all of them. In at least one embodiment, the first positively charged peptide segment is consisting of L-lysine.

The second hydrophobic peptide segment may be consisting of only L-phenylalanine, only L-tryptophan, only L-dopa, only L-tyrosine, only L-cysteine, only S-benzyl-L-cysteine, only S-methyl cysteine, or two, three, or more of them. In at least one embodiment, the second hydrophobic peptide segment is consisting of only S-benzyl-L-cysteine or only L-cysteine.

In at least one embodiment, the first positively charged peptide segment is L-lysine, and the second hydrophobic peptide segment is L-cysteine or S-benzyl-L-cysteine. And, in one embodiment, the block copolypeptide comprises Lys₅BnCys₅ (SEQ ID NO: 2), Lys₅Cys₅ (SEQ ID NO: 1), Lys₁₀BnCys₅ (SEQ ID NO: 5), Lys₁₀Cys₅ (SEQ ID NO: 19), Lys₁₅BnCys₅ (SEQ ID NO: 3), or Lys₁₅Cys₅ (SEQ ID NO: 20).

In at least one embodiment, the block copolypeptide is a single chain, or is branched, or star-shaped, for example, the block copolypeptide has the main structure shown in schematic FIG. 17. In FIG. 17, branches or arms of the branched or star-shaped block copolypeptide each independently have the first positively charged peptide segment and the second hydrophobic peptide segment, but the present disclosure is not limited in this way. In one embodiment, one of branches or arms has the first positively charged peptide segment and one of branches or arms has the second hydrophobic peptide segment. In another embodiment, one of branches or arms has both the first positively charged peptide segment and the second hydrophobic peptide segment, for example, the branched or star-shaped block copolypeptide is one selected from that shown in FIG. 20.

In at least one embodiment, the side chain of the block copolypeptide is modified with at least one selected from a group consisting of p-methoxybenzaldehyde, vanillin, cinnamaldehyde, catechol, indole, phenol, and phenyl, for example, the modified block copolypeptide has the main structure shown in schematic FIG. 18.

In one embodiment, the block copolypeptide is star-shaped and has multi-arms, wherein each of the arms comprises the first positively charged peptide segment and the second hydrophobic peptide segment. In one embodiment, a total number of the multi-arms is from 3 to 24, for example, 3, 4, 5, 6, 7, 8, 9, 10, 11, 12, 13, 14, 15, 16, 17, 18, 19, 20, 21, 22, 23, or 24.

The present application further provides a method for treating cancer, comprising administering an effective amount of a pharmaceutical composition to a subject in need thereof, wherein the pharmaceutical composition comprises the block copolypeptide of claim 1 and a pharmaceutically acceptable carrier.

In at least one embodiment, the effective amount is from 5 mg/kg to 10 mg/kg bodyweight of the subject, for example, 5, 5.5, 6, 6.5, 7, 7.5, 8, 8.5, 9, 9.5, 10 mg/kg bodyweight.

In at least one embodiment, the cancer is lung cancer, breast cancer, ovarian cancer, brain cancer, kidney cancer, oral cancer, esophageal stomach cancer, colon cancer, liver cancer, pancreas cancer, uterine cancer, endometrial cancer, cervical cancer, gastric cancer, skin cancer, testicular cancer, prostate cancer, and thyroid cancer. In one embodiment, the cancer is a lung cancer.

In one embodiment, the block copolypeptide suppresses the migration and adhesion of cancer cells. In another embodiment, the block copolypeptide inhibits the expression of E-cadherin in cancer cells. In yet another embodiment, the block copolypeptide suppresses the migration and adhesion of cancer cells and inhibits the expression of E-cadherin in cancer cells.

In at least one embodiment, the block copolypeptide is administered intravenously, intra-arterially, intra-peritoneally, intramuscularly, intradermally, intratumorally, orally, dermally, nasally, buccally, rectally, vaginally, by inhalation, or by topical to the subject.

In at least one embodiment, the method for treating cancer further comprises administering an effective amount of anti-cancer agent to the subject.

In at least one embodiment, the anti-cancer agent may be but not limited to cisplatin, oxaliplatin, nedaplatin, lobaplatin, or a combination thereof.

In one embodiment, the anti-cancer agent is administered with the block copolypeptide at the same time. In another embodiment, the anti-cancer agent is administered before or after the block copolypeptide. In yet another embodiment, the anti-cancer agent is administered in the same way as the block copolypeptide. In yet another embodiment, the anti-cancer agent is administered in a way different from the block copolypeptide.

The present disclosure also provides a method for increasing the proportion of Akkermansia muciniphila (Akk) in the gut, comprising administering an effective amount of a pharmaceutical composition to a subject in need thereof, wherein the pharmaceutical composition comprises the block copolypeptide of claim 1 and a pharmaceutically acceptable carrier.

In addition, the present disclosure provides a pharmaceutical composition comprising a block copolypeptide and a pharmaceutically acceptable carrier. The present disclosure provides use of a block copolypeptide in the manufacture of a medicament for treating cancer. The present disclosure provides a block copolypeptide for treating cancer.

In the second aspect, the present disclosure further provides a gene vector, comprising a block copolypeptide comprising a first positively charged peptide segment and a second hydrophobic peptide segment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will become more readily appreciated by reference to the following descriptions in conjunction with the accompanying drawings.

FIG. 1 is a schematic drawing of the possible signaling pathway in lung cancer cells of synthetic Lys₅Cys₅ (SEQ ID NO: 1) and Lys₅BnCys₅ (SEQ ID NO: 2).

FIGS. 2A-2C show effects of Lys₅Cys₅ (SEQ ID NO: 1) or Lys₅BnCys₅ (SEQ ID NO: 2) on cytotoxic and hemolytic activities. FIG. 2A is a bar graph showing A549 cell viability (%) response to treatment of peptides (Lys₅Cys₅ (SEQ ID NO: 1) or Lys₅BnCys₅ (SEQ ID NO: 2)) in the presence or absence of cisplatin (CP, 5 μM) for 24 h using a CCK-8 assay (n=4). FIG. 2B is a bar graph showing H1299 cell viability (%) response to treatment of peptides (Lys₅Cys₅ (SEQ ID NO: 1) or Lys₅BnCys₅ (SEQ ID NO: 2)) in the presence or absence of cisplatin (CP, 5 μM) for 24 h using a CCK-8 assay (n=4). FIG. 2C is a bar graph showing BEAS-2B cell viability (%) response to treatment of peptides (Lys₅Cys₅ (SEQ ID NO: 1) or Lys₅BnCys₅ (SEQ ID NO: 2)) in the presence or absence of cisplatin (CP, 5 μM) for 24 h using a CCK-8 assay (n=4). FIG. 2D is a bar graph (left panel) showing the percentage of hemolysis when the hemoglobin in the supernatant was analyzed using a microplate reader at 405 nm (n=4) and a table showing the percentage of hemolysis (right panel). The results obtained were subject to the Two-way ANOVA statistical analysis (FIGS. 2A-2D).

FIGS. 3A-3C show effects of Lys₅Cys₅ (SEQ ID NO: 1) or Lys₅BnCys₅ (SEQ ID NO: 2) on migration changes and adhesive ability in H1299 cells. FIG. 3A shows the results of a scratch wound healing assay on H1299 cells treated with Lys₅Cys₅ (SEQ ID NO: 1), Lys₅BnCys₅ (SEQ ID NO: 2), cisplatin or peptide combined with cisplatin (CP, 5 μM) to ascertain cell migration ability. The peptide solutions were added immediately right after the wounds were created. Wounds were evaluated at 24 h after peptide administration (scale bar=50 μm). FIG. 3B is a bar graph shows the percentage of wound-healing area quantified on the basis of the total cell area being 100%. (n=3). FIG. 3C is a bar graph shows the quantification of adhesive cells on the bottom of the wells (n=3). 1×10⁵ cells/well were seeded into 12-well plates and treated with Lys₅Cys₅ (SEQ ID NO: 1) or Lys₅BnCys₅ (SEQ ID NO: 2) for 24 hours. The adhesion ability to solid phase was determined by CCK-8 assay.

FIGS. 4A-4B show Lys₅Cys₅ (SEQ ID NO: 1) and Lys₅BnCys₅ (SEQ ID NO: 2) induced apoptotic signaling and adhesive ability changes in lung cancer cells. FIG. 4A shows results of Lys₅Cys₅ (SEQ ID NO: 1) or Lys₅BnCys₅ (SEQ ID NO: 2) induced caspase-3-dependent apoptosis in H1299 cells. Cells were treated with peptides for 24 hours, and total cell lysates were analyzed for E-cadherin, caspase-3, and PARP expression using immunoblot analysis. Histograms, which represent relative expression levels, were quantified using ImageJ software's densitometric analysis upon normalization on the basis of β-actin reference bands (n=3; *, #p<0.05, **, ##p<0.01, and ***, ###p<0.001). The * and #symbols represent the statistical significances of the caspase-3 vs. PARP-3-treated groups. FIG. 4B is a set of fluorescence images of A549 and H1299 cells treated with Lys₅Cys₅ (SEQ ID NO: 1) or Lys₅BnCys₅ (SEQ ID NO: 2) (4 μM) for 24 h using an inverted fluorescence microscopy (scale bar=100 μm). The white arrows indicate the apoptotic chromatin condensation of the nuclei.

FIGS. 5A-5F show results of Lys₅BnCys₅ (SEQ ID NO: 2) inhibited LL2 tumor growth and prolonged survival in tumor-bearing mice. FIG. 5A is a set of bioluminescence imaging conducted on day 12 upon the injection of D-luciferin in mice, and whole body images were shown with the photon flux scale. FIG. 5B is a bar graph showing quantification of bioluminescent imaging using an IVIS-200 system. FIG. 5C is a set of images of LL2 tumor-bearing mice treated with saline, Lys₅BnCys₅ (SEQ ID NO: 2) (5 mg/kg), Lys₅BnCys₅ (10 mg/kg), Lys₅BnCys₅ (5 mg/kg)+Cisplatin (2 mg/kg) and Cisplatin (2 mg/kg) at day 7, 10, 16 and 22 after the first treatment (n=8-10; *p<0.05, **p<0.01 and ***p<0.001). FIG. 5D is a graph showing tumor volume changes after intratumoral injection of saline, Lys₅BnCys₅ (SEQ ID NO: 2) (5 mg/kg), Lys₅BnCys₅ (10 mg/kg), Lys₅BnCys₅ (5 mg/kg)+Cisplatin (2 mg/kg) and Cisplatin (2 mg/kg) in LL2 tumor-bearing NOD-SCID mice (n=8). FIG. 5E is a graph showing changes of body weight were recorded starting from day 0 (n=8). FIG. 5F shows Kaplan-Meier survival curves with median survival times of tumor-bearing mice with different treatments. A log-rank test was used to evaluate the differences in survival (n=8). (b: Unpaired Student's t-test; d, e: two-way ANOVA).

FIGS. 6A-6D show results of Lys₅BnCys₅ (SEQ ID NO: 2) reduced LL2 tumor metastasis in lungs. FIG. 6A is an image of mice tumors. FIG. 6B shows the results of the measured tumor size and weight upon excised on day 32. FIGS. 6C-D shows the results of the number of pulmonary metastatic nodules analyzed using Lung tumor colony formation assay as indicated by black arrows. Scale bar=200 μm. (n=8). (b-c: Unpaired Student's t-test).

FIGS. 7A-7C shows microbial diversity of different treatment groups. FIG. 7A is a graph showing Lys₅BnCys₅ (SEQ ID NO: 2) treatment exhibited significant α-diversity (Shannon index) in compared with saline- and Lys₅BnCys₅+cisplatin-treated groups. FIG. 7B is a graph showing the β-diversity of the principal coordinate analysis (PCoA) demonstrated significant differences between these treatment groups in total microbial composition. FIG. 7C shows the relative abundance of rational taxonomic units (OTUs) at the phylum and species level in these groups (saline, Lys₅BnCys₅ (SEQ ID NO: 2), Lys₅BnCys₅+cisplatin-treated groups).

FIGS. 8A-8D show heatmap analysis of specific microbial constituents in Lys₅BnCys₅ (SEQ ID NO: 2)-treated group. FIGS. 8A-8B show Lys₅BnCys₅ (SEQ ID NO: 2) treatment increased the level of Verrucomicrobia as compared with pretreatment and saline groups. FIGS. 8C-8D show Lys₅BnCys₅ (SEQ ID NO: 2) treatment increased relative proportion of A. muciniphila as compared with pretreatment and saline groups.

FIG. 9 is a schematic drawing of Lys₅BnCys₅ (SEQ ID NO: 2) peptide interaction on cancer cell membrane and intestinal microbiota analysis of Lys₅BnCys₅ (SEQ ID NO: 2) peptide treatment.

FIGS. 10A-10C are ¹H NMR spectra of (a) Z-Lys₅, (b) Z-Lys₅BnCys₅, and (c) Lys₅BnCys₅ (SEQ ID NO: 2) peptides dissolved in TFA-d1, TFA-d1 and DMSO-d6, respectively.

FIGS. 11A-11B are ¹H NMR spectra of (a) Lys₅BnCys₅ (SEQ ID NO: 2) and (b) Lys₅Cys₅ (SEQ ID NO: 1) peptides dissolved in TFA-d1 and DMSO-d6, respectively.

FIG. 12A is a circular dichroism (CD) spectra of Lys₅BnCys₅ (SEQ ID NO: 2) and Lys₅Cys₅ (SEQ ID NO: 1) peptides. For CD analysis, peptide concentration was 0.1 mg mL⁻¹ in DI water. FIG. 12B is a FTIR spectra of Lys₅BnCys₅ (SEQ ID NO: 2) and Lys₅Cys₅ (SEQ ID NO: 1) peptides.

FIG. 13 is a plot showing small angle X-ray scattering (SAXS) pattern of Lys₅BnCys₅ (SEQ ID NO: 2) peptide and the peptide concentration was 0.5 wt % in DI water.

FIGS. 14A-14B are plots showing I₃/I₁ values of pyrene emission of (a) Lys₅BnCys₅ (SEQ ID NO: 2) and (b) Lys₅Cys₅ (SEQ ID NO: 1) peptide assemblies as a function of peptide concentration. The I3/I1 values were subtracted by 0.65.

FIG. 15 is a series of inverted fluorescence microscopy images (scale bar=100 μm) showing A549 and H1299 cells treated with peptides (4 μM) for 6 hours and stained with 1,1′-diethyl-4,4′-carbocyanine iodide (DCI, 1 μg/mL).

FIG. 16 is a series of immunochemical staining images showing H1299 cells treated with the Lys₅Cys₅ (SEQ ID NO: 1) or Lys₅BnCys₅ (SEQ ID NO: 2) for 24 hours, and stained with the anti-E-cadherin antibody (brown) and counterstained with hematoxylin (blue). CP, cisplatin (5 μM). Scale bar=100 μm.

FIG. 17 shows structures of linear and star-shaped block and graft copolypeptides.

FIG. 18 shows structures of linear and star-shaped block and graft copolypeptides.

FIG. 19 shows cell viability (%) treated with different copolypeptides at various concentrations.

FIG. 20 shows structures of star-shaped block copolypeptides including G2-PLL, G3-PLL, G2-PLA, G3-PLA, G3-PLL-MA, G3-PLL-VA, and G3-PLL-CA.

FIGS. 21A-21D show effects of copolypeptides on cytotoxic activities. FIG. 21A is a bar graph showing A549 cell viability (%) response to treatment of copolypeptides (G2-PLL₁₀ (having a side chain of SEQ ID NO: 13), G2-PLA₁₀ (having a side chain of SEQ ID NO: 14), G3-PLL₉ (having a side chain of SEQ ID NO: 15), G3-PLA₉ (having a side chain of SEQ ID NO: 16)). FIG. 21B is a bar graph showing H1299 cell viability (%) response to treatment of copolypeptides (G2-PLL₁₀ (having a side chain of SEQ ID NO: 13), G2-PLA₁₀ (having a side chain of SEQ ID NO: 14), G3-PLL₉ (having a side chain of SEQ ID NO: 15), G3-PLA₉ (having a side chain of SEQ ID NO: 16)). FIG. 21C is a bar graph showing A549 cell viability (%) response to treatment of copolypeptides (G3-PLL₉-CA_{0.3}, G3-PLL₉-VA_{0.25}, G3-PLL₉-MA_{0.2}). FIG. 21D is a bar graph showing H1299 cell viability (%) response to treatment of copolypeptides (G3-PLL₉-CA_{0.3}, G3-PLL₉-VA_{0.25}, G3-PLL₉-MA_{0.2}). FIG. 21E is a bar graph showing 3T3 cell viability (%) response to treatment of copolypeptides (G3-PLL₁₀-PLF₅, having a side chain of SEQ ID NO: 17). FIG. 21F is a bar graph showing RAW cell viability (%) response to treatment of copolypeptides (G3-PLL₁₀-PLF₅, having a side chain of SEQ ID NO: 17).

FIGS. 22A-22B show effects of copolypeptides on transfection efficiency. FIG. 22A is a series of images showing the transfection efficiencies of various copolypeptides. FIG. 22B is a bar graph showing effects of various copolypeptides on transfection efficiency (%).

FIG. 23A is a bar graph showing H1299 cell viability (%) response to treatment of dendrimer/pDNA complexes. FIG. 23B is a bar graph showing 293T cell viability (%) response to treatment of dendrimer/pDNA complexes.

FIG. 24 shows the results of a scratch wound healing assay on cancer cells treated with various copolypeptides (G3-PLL₉ (having a side chain of SEQ ID NO: 15), G3-PLA₉, G3-PLL₉-CA_{0.3}, G3-PLL₉-VA_{0.25}, and G3-PLL₉-MA_{0.2}). Images were visualized at 0 hr and 24 hr at a magnification of 100×. Copolypeptides treatment inhibits the migration ability of cancer cells.

FIG. 25A is a bar graph showing wound area (%) response to treatment of copolypeptides (G3-PLL₉, having a side chain of SEQ ID NO: 15), G3-PLA₉ (having a side chain of SEQ ID NO: 16), G3-PLL₉-CA_{0.3}, G3-PLL₉-VA_{0.25}, and G3-PLL₉-MA_{0.2}). FIG. 25B shows the cell migration (%) response to treatment of copolypeptides (G3-PLL₉, having a side chain of SEQ ID NO: 15), G3-PLA₉ (having a side chain of SEQ ID NO: 16), G3-PLL₉-CA_{0.3}, G3-PLL₉-VA_{0.25}, and G3-PLL₉-MA_{0.2}).

FIG. 26 is a set of images showing apoptosis of H1299 cancer cells response to treatment of copolypeptides (G3-PLL₉, having a side chain of SEQ ID NO: 15), G3-PLA₁₀ (having a side chain of SEQ ID NO: 18), G3-PLL₉-CA_{0.3}, G3-PLL₉-VA_{0.25}, and G3-PLL₉-MA_{0.2}) visualized at 0′, 10′, 40′ and 50′ (min).

FIG. 27 is a set of images showing apoptosis of 293T, H1299, and A549 cancer cells response to treatment of G3-PLL₉-VA_{0.25} visualized at 0′, 10′, 40′ and 50′.

FIG. 28 is a set of immunofluorescence staining images showing H1299 cancer cells about apoptosis treated with G3-PLL₉-VA_{0.25} at 0 μM, 0.05 μM, 0.1 μM, and 0.2 μM. Cancer cells about apoptosis are stained with DAPI and Nile red.

FIG. 29 is a set of immunofluorescence staining images showing A549 cancer cells about apoptosis treated with G3-PLL₉-VA_{0.25} at 0 μM, 0.05 μM, 0.1 μM, and 0.2 μM. Cancer cells about apoptosis are stained with DAPI and Nile red.

### DETAILED DESCRIPTIONS OF THE PREFERRED EMBODIMENTS

The following embodiments are provided to illustrate the present disclosure in detail. A person having ordinary skill in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

As used herein, the singular forms “a,” “an,” and “the” include plural referents unless expressly and unequivocally limited to one referent. The term “or” is used interchangeably with the term “and/or” unless the context clearly indicates otherwise.

The terms “short peptide(s)” are used herein to designate a series of amino acid residues, connected one to the other typically by peptide bonds between the alpha-amino and carbonyl groups of the adjacent amino acids. The peptides are preferably 10 or less amino acids in length, and 2 or more amino acids in length, for example, 2, 3, 4, 5, 6, 7, 8, 9 or 10 amino acids in length.

The terms “peptide(s),” “polypeptide(s)” and “copolypeptide(s)” are used herein to designate a series of amino acid residues that have much more amino acid residues than “short peptide(s),” such as 10 to 40 amino acids in length, 10 to 30 amino acids in length or 10 to 25 amino acids in length, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length. “Copolypeptide(s)” refers to a long amino acid sequence comprising two or more types of amino acids.

Peptides, polypeptides, or copolypeptides (hereinafter referred to as peptides) in the present application may include the standard 20 α-amino acids that are used in protein synthesis by cells (i.e. natural amino acids), as well as non-natural amino acids (may be found in nature, but not used in protein synthesis by cells, e.g., orithine, citrulline, and sarcosine, or may be chemically synthesized), amino acid analogs, and peptidomimetics. The amino acids may be D- or L-optical isomers. The peptides may be formed by a condensation or coupling reaction between the α-carbon carboxyl group of one amino acid and the amino group of another amino acid. The terminal amino acid at one end of the chain (amino terminal) therefore has a free amino group, while the terminal amino acid at the other end of the chain (carboxy terminal) has a free carboxyl group. Alternatively, the peptides may be non-linear, branched peptides or cyclic peptides. Moreover, the peptides may optionally be modified or protected with a variety of functional groups or protecting groups, including on the amino and/or carboxy terminus.

The amino acid residues in the peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The peptides are therapeutic themselves and serve as drugs or therapeutic agents, or serve as active compounds in the pharmaceutical composition. Or, the peptides may serve as carriers, vehicles, or vectors for other drugs or therapeutic agents. Other drugs or therapeutic agents, may be anti-cancer agents, genes, or combination thereof, for example, cisplatin, oxaliplatin, nedaplatin, and/or lobaplatin.

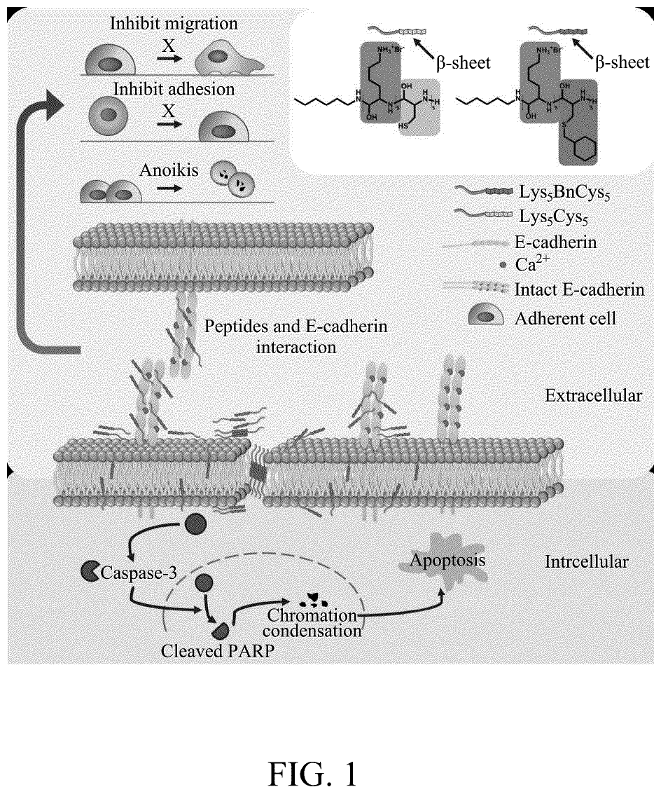

FIG. 1 shows the peptides, specifically the block copolypeptides (e.g., Lys₅BnCys₅ (SEQ ID NO: 2) and Lys₅Cys₅ (SEQ ID NO: 1)) comprising a first positively charged peptide segment and a second hydrophobic peptide segment, interact with the targeted cells such as cancer cells (e.g., lung cancer cells). In at least one embedment of the present disclosure, the peptides damage integrity of the cell membrane of the targeted cells, and/or interrupt expression or intercalation of E-cadherin on the cell membrane of the targeted cells, especially interfering with the assembly of E-cadherin and calcium ions to form intact E-cadherin through their coil-sheet structure, so that migration and adhesion of the targeted cells are inhibited and the anoikis thereof is promoted. Further, the caspase-3, poly(ADP-ribose) polymerase (PARP), and chromation condensensation in the targeted cells are triggered and activated to promote apoptosis thereof. The peptides of the present disclosure may achieve therapeutic effects based on this theory (i.e., simultaneously through intracellular and extracellular signal pathway), or it is believed that therapeutic effects may not be bound by this theory. In FIG. 1, although segments consisting of L-lysine and segments consisting of L-cysteine or S-benzyl-L-cysteine are used to form coil shape and β-sheet shape, respectively, other amino acids can be used.

The peptides may contain or may be modified to contain, functional groups to which a water-soluble polymer may be attached, either directly or through a spacer moiety or linker. Functional groups include, but are not limited to, the N-terminus of the peptide, the C-terminus of the peptide, and any functional groups on the side chain of an amino acid, e.g. lysine, cysteine, histidine, aspartic acid, glutamic acid, tyrosine, arginine, serine, methionine, and threonine, present in the peptides.

The terms “subject,” “individual,” or “patient” are used interchangeably herein and refer to a vertebrate, preferably a mammal. Mammals include, but are not limited to, murines, rodents, simians, humans, farm animals, sport animals, and pets.

“Optional” or “optionally” means that the subsequently described circumstance may or may not occur, so that the description includes instances where the circumstance occurs and instances where it does not.

The term “about” as used herein when referring to the numerical value is meant to encompass variations of 20%, ±10%, ±5%, ±1%, ±0.5%, or ±0.1% from the numerical value. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations.

As used herein, the terms “comprise,” “comprising,” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

Short peptides include, but are not limited to, fragments, variants, and derivatives of the peptides. The short peptides also include any and all combinations of these modifications. In a non-limiting example, a short peptide may be a fragment of the peptides as disclosed herein or even the fragment having one or more amino acid substitutions. Thus it will be understood that any reference to a short peptide derived from a peptide is not limited to having only that particular modification, but rather encompassing that particular modification and optionally any other modifications.

The short peptides may arise by natural processes (e.g. processing and other post-translational modifications) or may be made by chemical modification techniques. Such modifications are well-known to those of skill in the art.

The short peptides may have a single alteration or multiple alterations relative to the parent peptide. Where multiple alterations are present, the alterations may be of the same type or different type.

Furthermore, modifications can occur anywhere in the peptides, including the peptide backbone, the amino acid side-chains, and the N- or C-termini.

An “effective amount” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “anti-cancer activity” refers to the peptide being capable of changing the function or metabolism of a target cancer cell, such as influencing reproduction, growth, toxin production, subsistence, etc., but it is not limited thereto. In one embodiment, anticancer activity refers to inhibiting cancer cell growth. Moreover, in one specific embodiment, anticancer activity refers to the peptide being capable of killing at least one kind of cancer cell.

As used herein, the term “wound-healing” in the present disclosure may be continuous, dynamic and complicated processes, which may comprise, but are not limited to, cell proliferation, cell migration, etc. In one embodiment, the phrase “wound-healing promoting” used in the present disclosure also may refer to “enhancing cell proliferation” or “enhancing cell migration”, but it is not limited thereto.

The effect of wound-healing promoting of the pharmaceutical composition of the present disclosure also can refer to effect of “enhancing cell proliferation”, “enhancing cell migration”, etc., but is not limited thereto.

The pharmaceutical composition of the present disclosure may be administered intravenously, intra-arterially, intra-peritoneally, intramuscularly, intradermally, intratumorally, orally, dermally, nasally, buccally, rectally, vaginally, by inhalation, or by topical administration.

The pharmaceutical composition of the present disclosure may be administered to a plant or an animal, etc. The preceding animal may be a fish, a bird, a mammal, etc., but it is not limited thereto. Examples of the mammal may include, but is not limited to, a cat, a dog, a bovine, a horse, a pig, a human, etc. In one embodiment, the pharmaceutical composition of the present disclosure may be administered to a human.

The copolypeptides of the present disclosure may be synthesis by the ring-opening polymerization (ROP) method. For example, amino acids suitable for synthesizing the segment having desired property (i.e., positively charged or hydrophobic) are selected and reacted to form N-carboxyanhydrides (NCAs). Optionally, some amino acids require an additional side chain protection step (for example, using benzyloxycarbonyl group (Z group)) and such step is performed prior to the NCA formation step. ROP is then carried out by amine initiators and NCAs to form the desired copolypeptides. If a side chain protection step has been performed previously, an additional side chain deprotection step is required after copolypeptides formation.

In one embodiment, L-lysine (suitable for synthesizing a positively charged segment) and S-Benzyl-L-cysteine (suitable for synthesizing a hydrophobic segment) are selected and L-lysine is reacted with benzyl chloroformate to form Z-Lys (i.e., L-lysine protected by a Z group). Then, Z-Lys and BnCys are reacted to form Z-Lys-NCAs and BnCys-NCAs, respectively. Z-LysnmBnCysn copolypeptide is synthesized by ROP of Z-Lys-NCAs and BnCys-NCAs using hexylamine as the initiator and LysₘBnCysₙ copolypeptide was then obtained by deprotection of the Z group. m and n range from 1 to 50, from 1 to 40, from 1 to 30, or from 1 to 20.

In one embodiment, LysₘCysₙ copolypeptide is derived from Z-LysₘBnCysₙ copolypeptide or LysₘBnCysₙ copolypeptide. Here, Z-LysₘBnCysₙ copolypeptide undergoes a deprotection step to form LysₘBnCysₙ, and further reacts with an agent that is able to leave the S-benzyl group on BnCys segment to form LysₘCysₙ.

In one embodiment, LysₘBnCysₙ is Lys₅BnCys₅ (SEQ ID NO: 2) to Lys₂₀BnCys₅ (SEQ ID NO: 21), such as Lys₅BnCys₅ (SEQ ID NO: 2), Lys₁₀BnCys₅ (SEQ ID NO: 5), Lys₁₅BnCys₅ (SEQ ID NO: 3), and Lys₂₀BnCys₅ (SEQ ID NO: 21). In one embodiment, LysₘCysₙ is Lys₅Cys₅ (SEQ ID NO: 1) to Lys₂₀Cys₅ (SEQ ID NO: 22), such as Lys₅Cys₅ (SEQ ID NO: 1), Lys₁₀Cys₅ (SEQ ID NO: 19), Lys₁₅Cys₅ (SEQ ID NO: 20), and Lys₂₀Cys₅ (SEQ ID NO: 22).

Exemplary embodiments of the present disclosure are further described in the following examples, which should not be construed to limit the scope of the present disclosure.

### Characterization of Copolypeptides

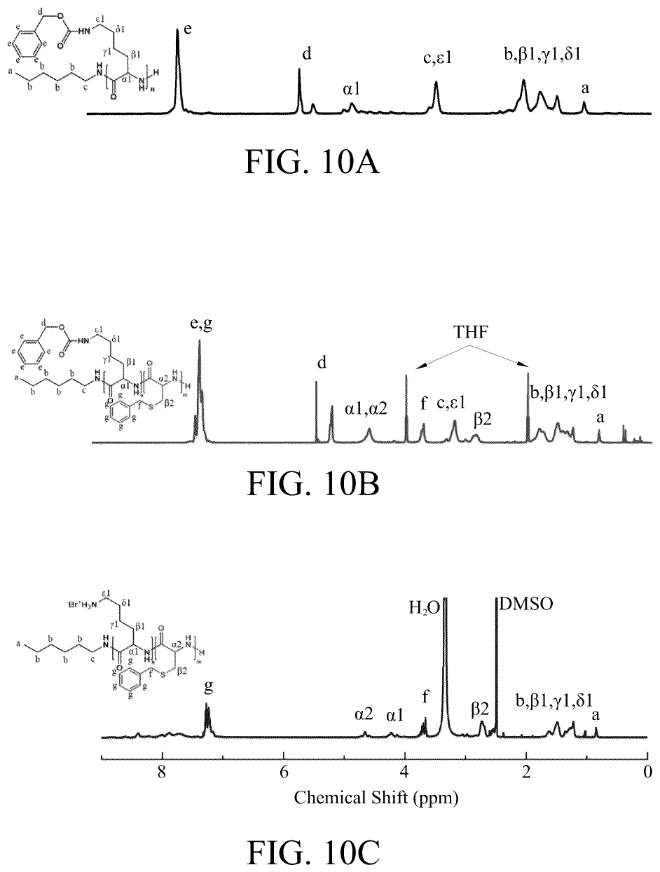

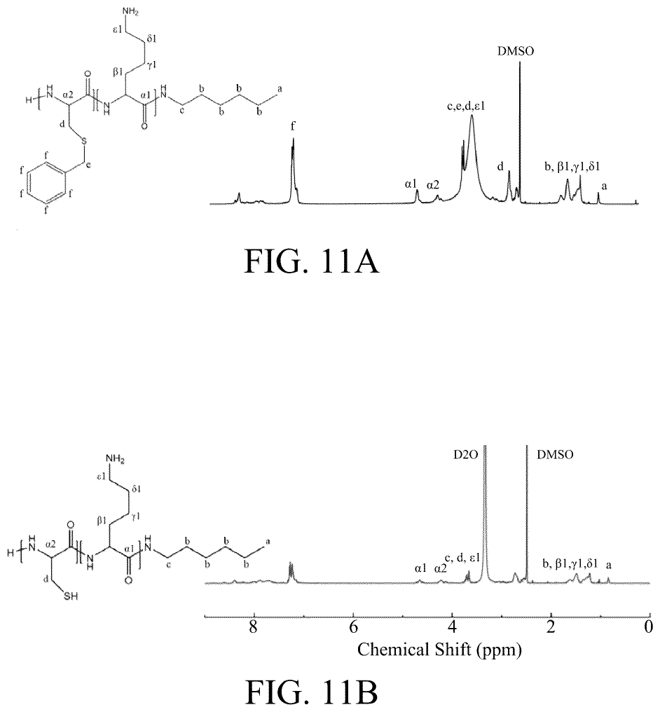

The chain lengths of two segments on Z-Lysₘ, Z-LysₘBnCysₙ, and LysₘBnCysₙ of the present disclosure were derived by the integral ratios of the protons on the initiator (CH₃CH₂—), the protons on Z-Lys (—(CH₂)₃CH₂—), and the protons of methyl group on BnCys (—CH₂SCH₂— or —CH₂SCH₂—) as shown in FIGS. 10A-10C. The integral ratio of initiator, Z-Lys, and BnCys of the present disclosure was calculated to be 1:5: 5, consistent with the feed molar ratio (1:5: 5). Matrix-assisted laser desorption ionization-time of flight mass spectrometry (MALDI-TOF MS) analysis showed that the derived number-averaged molecular weight (Mₙ) of the side-chain protected peptide was 2300 g mol⁻¹, with a low molecular weight distribution (M_{w}/Mₙ 1.08), which is comparable with theoretical values (2375 g mol-1). Lys₅BnCys₅ (SEQ ID NO: 2) was obtained by removing only the Z group on the Z-Lys segment of Z-Lys₅BnCys₅ using HBr. As shown in FIG. 10C and FIG. 11A, ¹H NMR analysis of the Lys₅BnCys₅ (SEQ ID NO: 2) confirmed that the benzyl group on BnCys segment was intact.

Lys₅Cys₅ (SEQ ID NO: 1) was obtained by removing both the Z and benzyl groups on respective Z-Lys and BnCys segments of Lys₅BnCys₅ (SEQ ID NO: 2) using HBr and trimethylsilyl iodide (TMSI, Alfa Aesar). ¹H NMR analysis of the Lys₅Cys₅ (SEQ ID NO: 1) revealed that the percentage of residual Z and benzyl groups was below 12% (FIG. 11B), suggesting the removal of protecting groups on Z-Lys and BnCys segments.

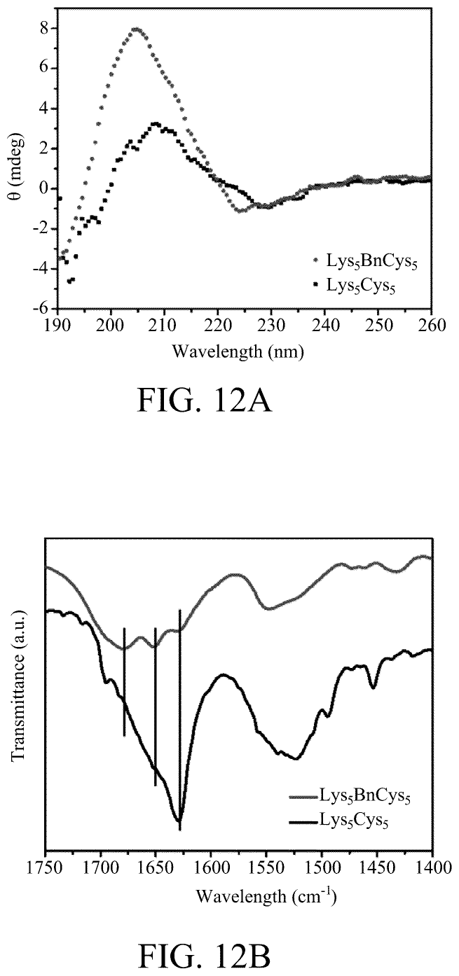

The chain conformations adopted by the two copolypeptides Lys₅BnCys₅ (SEQ ID NO: 2) and Lys₅Cys₅ (SEQ ID NO: 1) at neutral condition were characterized using CD and FTIR analyses. The BeStSel software (Beta Structure Selection) was used to compute the percentages of peptide chain conformation by fitting the CD spectra. The two copolypeptides adopted a mixture of random coil and β-sheet/turn conformations (FIG. 12A and Table 1), consistent with the results from the FTIR analysis showing the characteristic peaks of β-sheet, random coil, and R-turn at about 1626, 1651, and 1677 cm 1, respectively (FIG. 12B).

TABLE 1
Percentages of various secondary conformations adopted by the
copolypeptides

| Copolypeptides | Random coil (%) | ß-sheet/turn (%) |
|---|---|---|
| Lys₅BnCys₅ (SEQ ID NO: 2) | 23.8 | 76.2 |
| Lys₅Cys₅ (SEQ ID NO: 1) | 32.0 | 68.0 |

Both Cys₅ and BnCys₅ segments form intermolecular β-sheets/turns via hydrophobic and hydrogen bonding interactions. And, the percentages of β-sheet/turn conformation adopted by the copolypeptides of the present disclosure were much higher than the fraction of sheet-like BnCysₙ or Cysₙ segment. The percentage of β-sheet/turn conformation adopted by Lys₅BnCys₅ (SEQ ID NO: 2) was slightly higher than that adopted by Lys₅Cys₅ (SEQ ID NO: 1), which could be due to the presence of benzyl group.

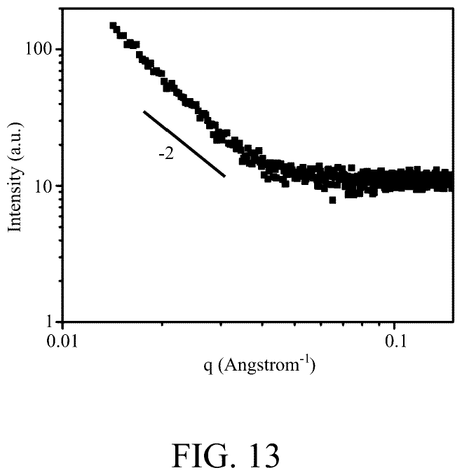

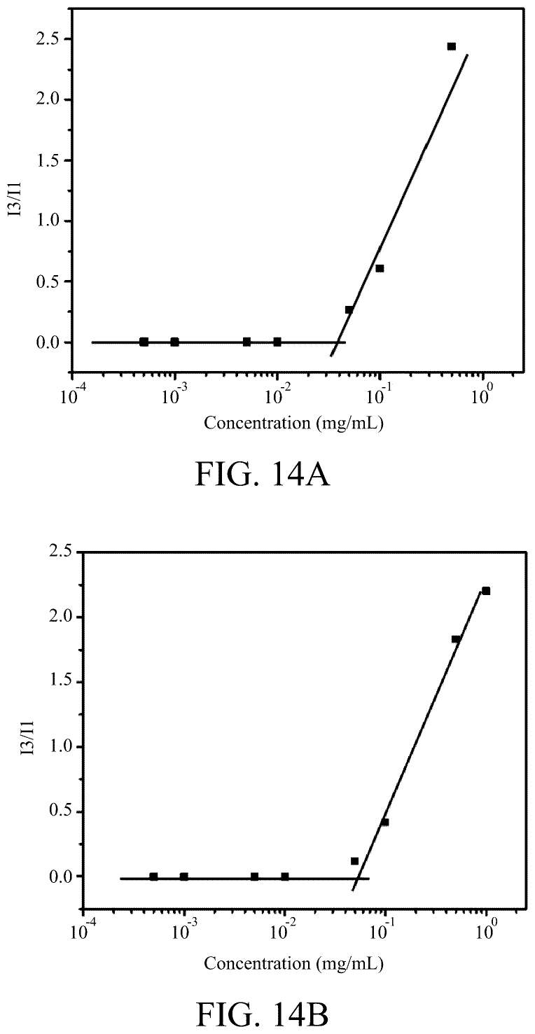

In this example, the coil poly(L-lysine) tethered with the hydrophobic, sheet-like peptide segment, resulting in the copolypetides tending to form bilayer assemblies in an aqueous solution. In FIG. 13, SAXS patterns of the copolypeptide solution revealed the formation of bilayer assemblies, evidenced by the scattering intensity (I(q)) with characteristic I(q)∝q⁻² at the low scattering vectors (q). The critical aggregation concentration (cac) of Lys₅BnCys₅ (SEQ ID NO: 2) and Lys₅Cys₅ (SEQ ID NO: 1) was determined to be approximately 0.035 and 0.05 mg mL⁻¹ (16.63 and 30.21 μM), respectively (FIGS. 14A-14B). The results showed that these two copolypeptides could self-assemble to form bilayers once the peptide concentration was above its cac value, while Lys₅BnCys₅ (SEQ ID NO: 2) exhibits better packing ability than Lys₅Cys₅ (SEQ ID NO: 1), evidenced by the cac value of Lys₅BnCys₅ (SEQ ID NO: 2) lower than that of Lys₅Cys₅ (SEQ ID NO: 1). Upon forming bilayer assemblies, the confined Lys₅ segment would undergo coil-sheet/turn conformational change, resulting in high percentages of β-sheet/turn conformation.

### Example 1: Lys₅BnCys₅ (SEQ ID NO: 2) and Lys₅Cys₅ (SEQ ID NO: 1) Enhance Cytotoxic and Hemolytic Activities

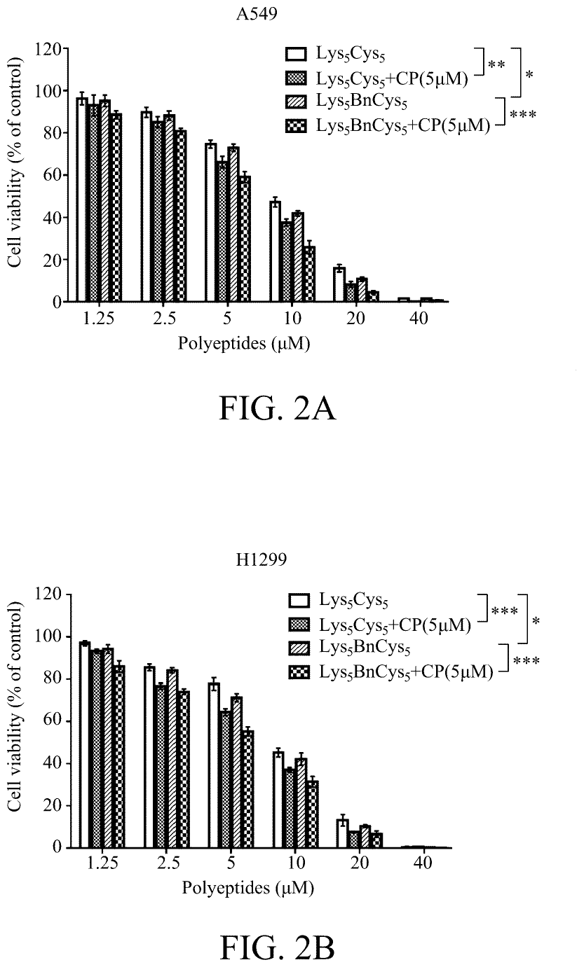

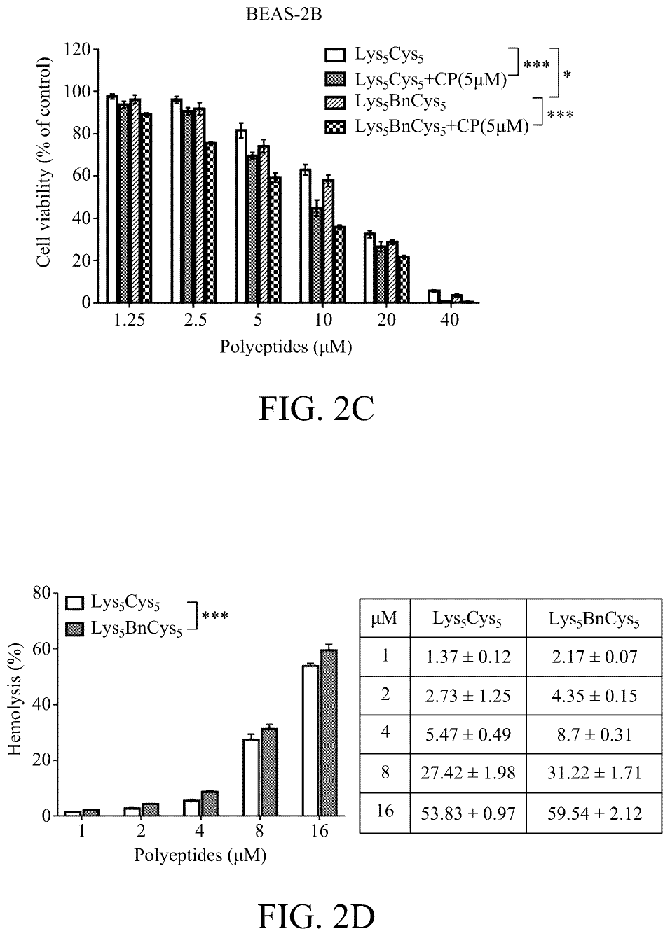

In order to determine whether Lys₅BnCys₅ (SEQ ID NO: 2) would enhance cytotoxicity in cancer cells of the present disclosure, A549 and H1299 cancer cells and BEAS-2B normal cells were treated with Lys₅Cys₅ (SEQ ID NO: 1) or Lys₅BnCys₅ (SEQ ID NO: 2) for 24 h. As shown in FIGS. 2A-2B, both cancer cells (A549 and H1299) treated with Lys₅BnCys₅ (SEQ ID NO: 2) exhibited lower IC₅₀ values than the Lys₅Cys₅ (SEQ ID NO: 1)-treated ones (Table 2).

TABLE 2
IC₅₀ of copolypeptide-treated cancer and normal cells.

| | Cell line [symbol] | | | |
|---|---|---|---|---|
| IC₅₀ (μm) [symbol] | A549 [symbol] | H1299 [symbol] | BEAS-2B [symbol] | [symbol] |
| LyssCys₅ [symbol] | 9.71 [symbol] | 9.73 [symbol] | 13.93 [symbol] | [symbol] |
| LyssCys₅ + Cisplatin (50 nM) [symbol] | 8.02 [symbol] | 7.8 [symbol] | 9.79 [symbol] | [symbol] |
| LyssBnCys₅ [symbol] | 8.85 [symbol] | 8.99 [symbol] | 12.49 [symbol] | [symbol] |
| LyssBnCys₅ + Cisplatin (50 nM) [symbol] | 6.7 [symbol] | 6.92 [symbol] | 7.32 [symbol] | [symbol] |

Moreover, after treatment with these two copolypeptides, normal cells (BEAS-2B) presented lower cytotoxicity than either cancer cell (FIG. 2C). This phenomenon may also be treated with peptide fibril assemblies.

In addition, the addition of low dose cisplatin (CP) enhanced peptide-induced cytotoxicity in these cells (FIGS. 2A-2C). To further confirm the cytotoxicity toward suspension cells and the feasibility for in vivo application, human red blood cells (RBCs) were co-incubated with peptides for 1 h. As shown in FIG. 2D, RBCs treated with Lys₅BnCys₅ (SEQ ID NO: 2) exhibited more hemolytic activity compared to Lys₅Cys₅ (SEQ ID NO: 1)-treated ones. Taken together, these results reveal that Lys₅Cys₅ (SEQ ID NO: 1) and Lys₅BnCys₅ (SEQ ID NO: 2) both exhibited cytotoxicity in adherent and suspension cells and Lys₅BnCys₅ (SEQ ID NO: 2) exhibited much enhanced cytotoxicity.

### Example 2: Lys₅BnCys₅ (SEQ ID NO: 2) and Lys₅Cys₅ (SEQ ID NO: 1) Inhibit the Migration and Adhesion of Lung Cancer Cells

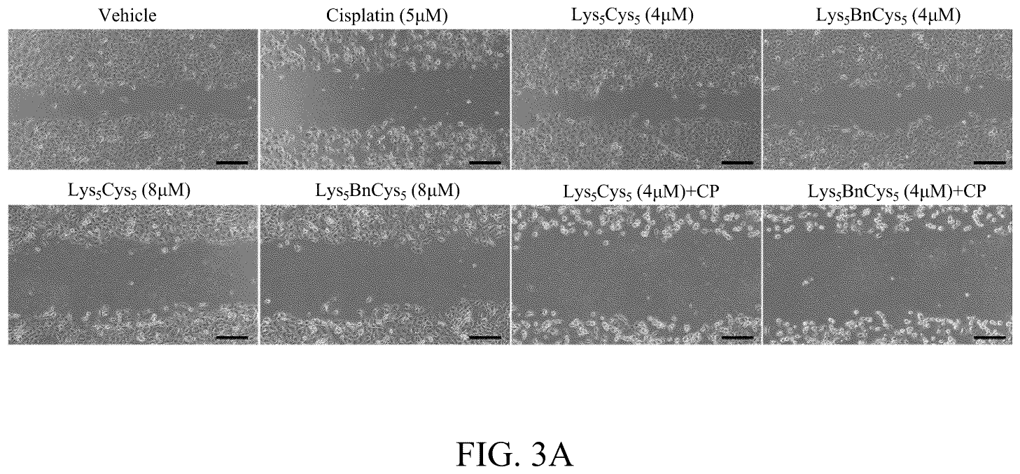

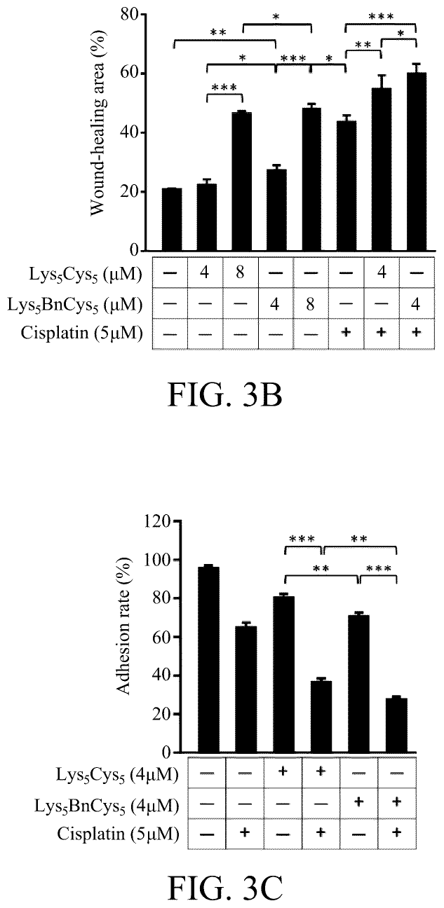

In order to explore whether the copolypeptides could affect cell migration, wound healing assay was performed. After a scratch line was induced, H1299 cells were treated with copolypeptides or additionally treated with cisplatin (CP) for 24 h. As shown in FIGS. 3A-3B, both copolypeptides inhibited cell migration and the addition of low dose cisplatin (5 μM) enhanced these effects. Moreover, Lys₅BnCys₅ (SEQ ID NO: 2) treatment exhibited larger wound healing area than Lys₅Cys₅ (SEQ ID NO: 1) under the same concentration.

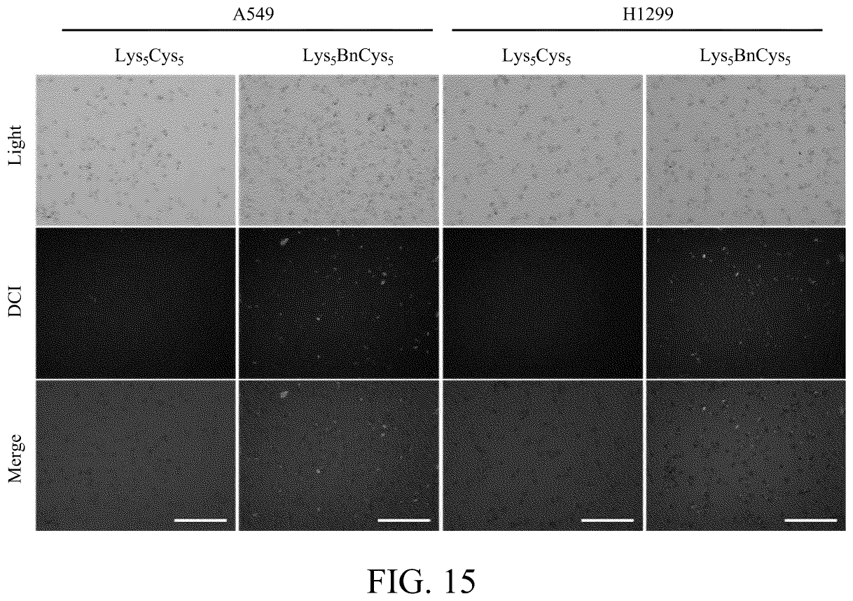

To further verify whether the benzyl group on Lys₅BnCys₅ (SEQ ID NO: 2) was involved in regulating cell migration processes, cell membranes were stained with 1,1′-diethyl-4,4′-carbocyanine iodide which, like most lipophilic carbocyanine dyes, is used for phospholipid bilayer labeling to track membrane changes. As shown in FIG. 15, A549 and H1299 treated with Lys₅BnCys₅ (SEQ ID NO: 2) for 6 h could be highly stained with 1,1′-diethyl-4,4′-carbocyanine iodide, but not the Lys₅Cys₅ (SEQ ID NO: 1) treated ones. To further confirm whether Lys₅BnCys₅ (SEQ ID NO: 2) could interfere with cell adhesion, cell adhesion rates were determined after treatment with the copolypeptides. As shown in FIG. 3C, Lys₅BnCys₅ (SEQ ID NO: 2) affected cell adhesion markedly in comparison to Lys₅Cys₅ (SEQ ID NO: 1), which was also promoted by adding low-dose cisplatin (FIG. 3C). These results suggest that Lys₅BnCys₅ (SEQ ID NO: 2) further integrated into the membranes and the membranes lost their integrity, as can be seen with 1,1′-diethyl-4,4′-carbocyanine iodide staining. The loss of integrity caused by Lys₅BnCys₅ (SEQ ID NO: 2) decreased cell migration. Thus, the benzyl group on Lys₅BnCys₅ (SEQ ID NO: 2) seems to play a role in the suppression of cell migration.

### Example 3: Lys₅BnCys₅ (SEQ ID NO: 2) and Lys₅Cys₅ (SEQ ID NO: 1) Affect E-Cadherin Expression and Induces Apoptotic Signaling

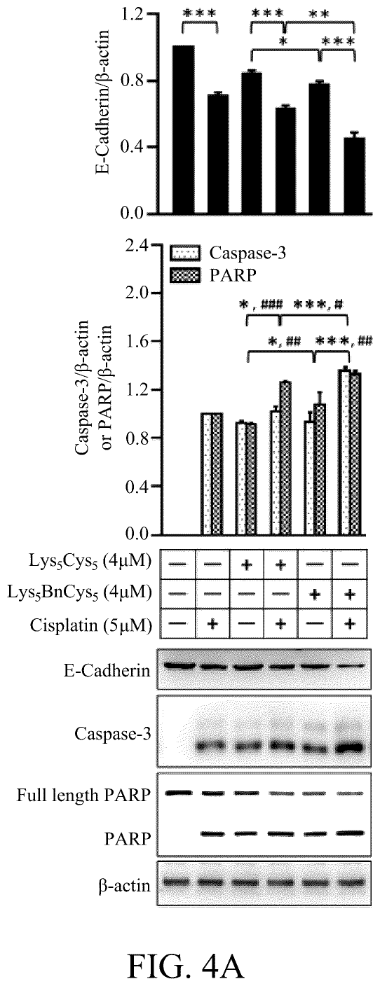

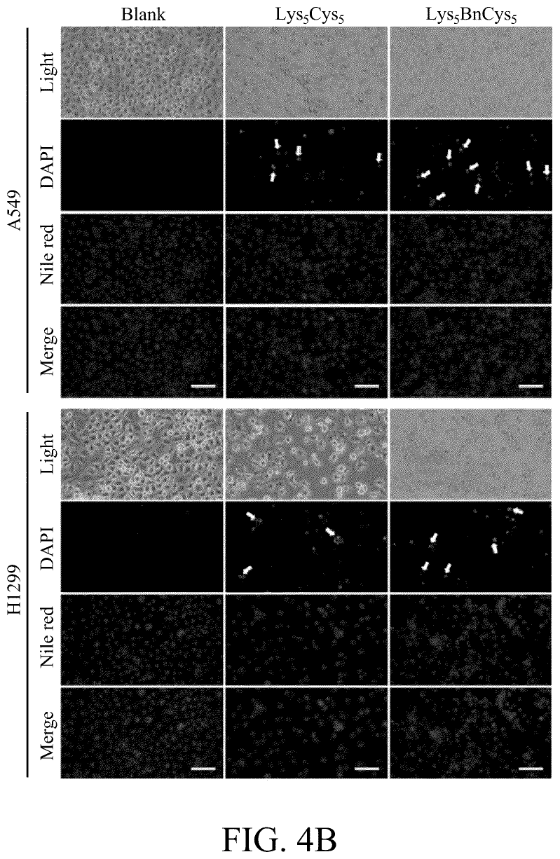

In the present disclosure, E-cadherin is one of the crucial molecules in cell-cell adhesion, participating in cancer progression. To verify whether Lys₅BnCys₅ (SEQ ID NO: 2) and Lys₅Cys₅ (SEQ ID NO: 1) could affect cell adhesion and apoptotic signaling, E-cadherin, caspase-3, and PARP expressions in H1299 of the present disclosure were evaluated after treatment with the copolypeptides for 24 h. As shown in FIG. 4A, Lys₅BnCys₅ (SEQ ID NO: 2) and Lys₅Cys₅ (SEQ ID NO: 1) reduced E-cadherin expression and triggered caspase-3, and PARP expression significantly and the effects of Lys₅BnCys₅ (SEQ ID NO: 2) are stronger than Lys₅Cys₅ (SEQ ID NO: 1).

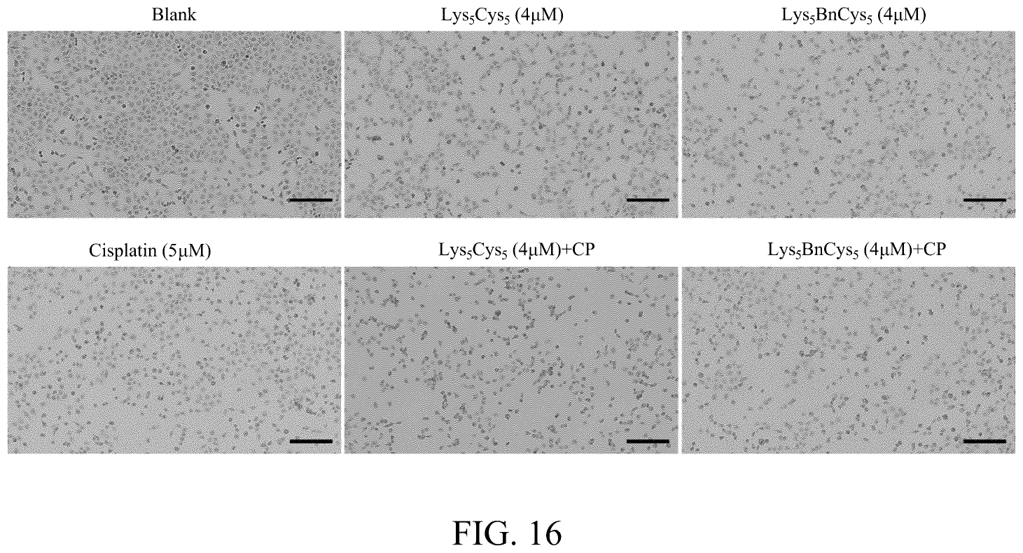

The location of cellular E-cadherin was also stained to evaluate cell-cell interaction after peptide treatment. Cells treated with Lys₅BnCys₅ (SEQ ID NO: 2) became rounder (i.e., detached) than Lys₅Cys₅ (SEQ ID NO: 1) treated ones, as seen by the membranous E-cadherin stained brown (FIG. 16). Downregulation of E-cadherin and upregulation of apoptotic signaling were both enhanced by adding copolypeptides and low-dose cisplatin (5 μM).

The DNA condensation step of apoptotic signaling was also observed starting in FIG. 4B and was consistent with western blot results (FIG. 4A). Lys₅BnCys₅ (SEQ ID NO: 2) treatment also resulted in much flatter cells, more leakage of intracellular contents, and serious loss of the original saturation of the cells (FIG. 4B, Light image). In the Lys₅BnCys₅ (SEQ ID NO: 2) treated group, cell membranes showed tears and clustering as observed by Nile red staining (FIG. 4B). These results were consistent with the migration assay, corroborating that the benzyl group enhanced the influences of copolypeptides on cell-cell adhesion, cell migration, and apoptosis. Thus, Lys₅BnCys₅ (SEQ ID NO: 2) and Lys₅Cys₅ (SEQ ID NO: 1) were considered the effective apoptotic inducer and migration inhibition, while Lys₅BnCys₅ was more effective one.

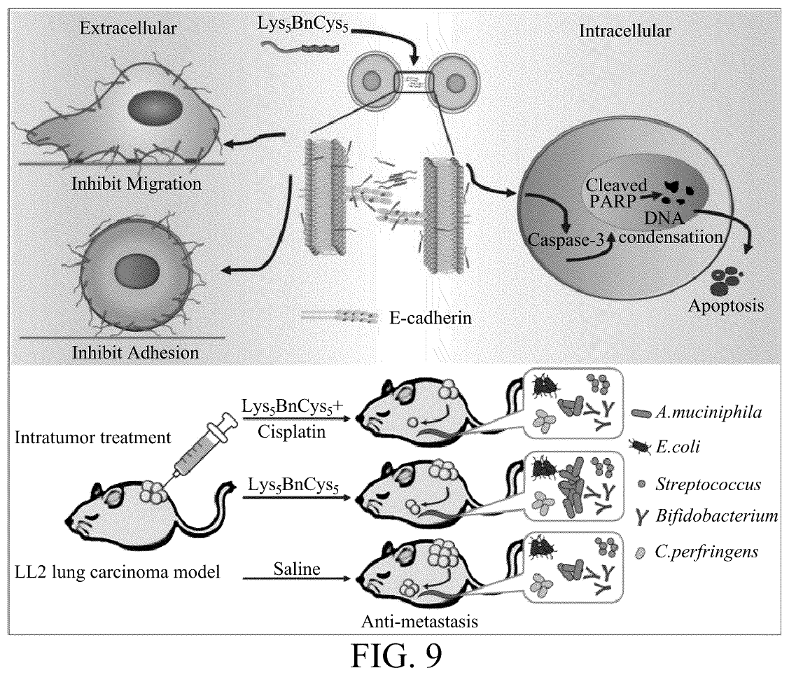

One having ordinary skills in the art will understand that in some instances, E-cadherin expression plays a complicated role in the process of promoting and inhibiting tumor metastasis at different stages. However, a molecule that could inhibit E-cadherin-mediated survival in metastatic cancers was suggested as a potential therapeutic agent. The present disclosure provides that the copolypeptide treatment suppresses the migration and adhesion of cancer cells (FIG. 3), also provides that the copolypeptides interact with the cell membrane and affect normal E-cadherin expression through electrostatic, hydrophobic, and hydrogen bonding interactions (FIGS. 4, 15 and 16). It is believed that the cationic Lys and bulky BnCys or Cys segments herein bind with the negatively charged cadherin molecules and act as a spacing moiety to prevent the packing of E-cadherin, respectively. Moreover, the binding of peptides with cell membrane or E-cadherin molecule of the present disclosure drastically changes the peptide amphiphilicity, facilitating the packing of peptide chains onto cell membrane or between E-cadherin molecules. In the present disclosure, it was considered that due to the aforementioned influences of copolypeptides on E-cadherin of target cells and damaged integrity of target cell membrane caused by the intercalation of copolypeptides into the lipid bilayer, the migration and adhesion of target cells were inhibited while caspase-3 and PARP in target cells are triggered and activated, thereby inducing apoptosis (FIGS. 9 and 1).

### Example 4: Lys₅BnCys₅ (SEQ ID NO: 2) has Antitumor Effects in Syngeneic Mouse Model

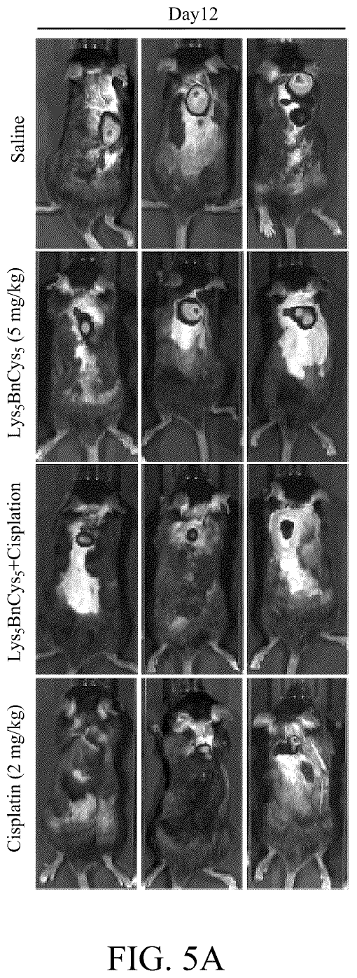

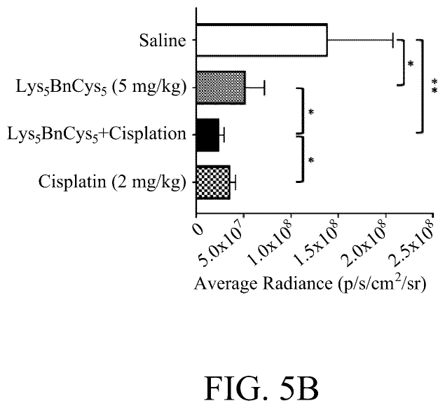

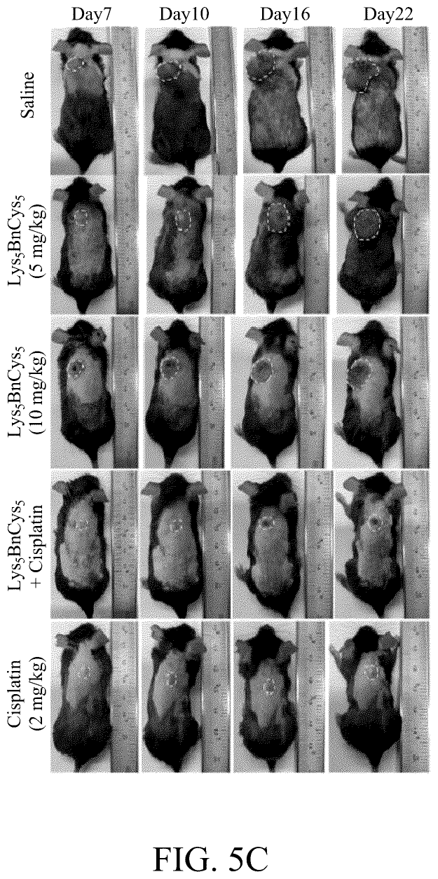

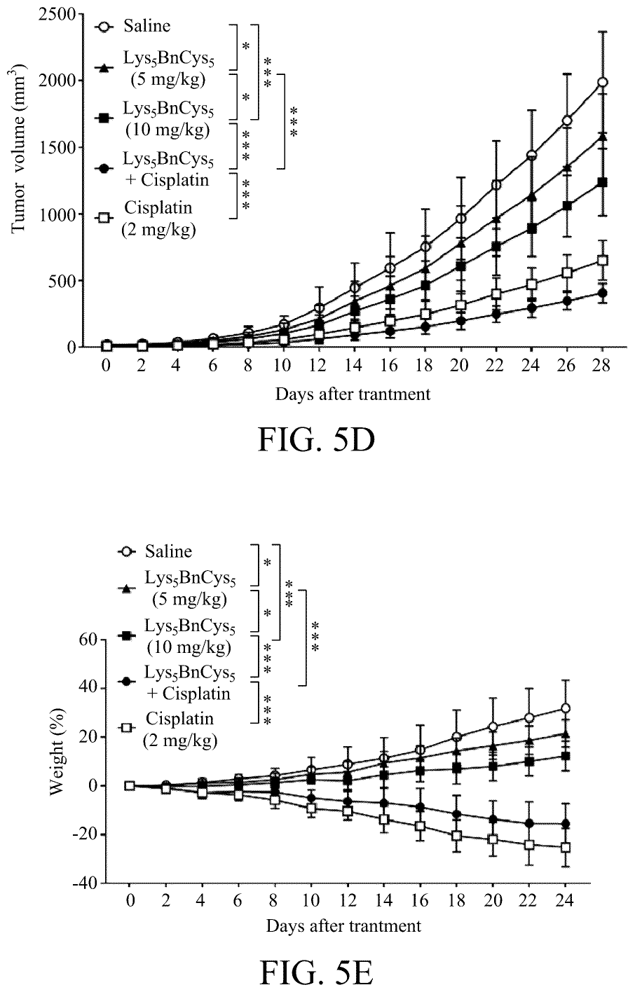

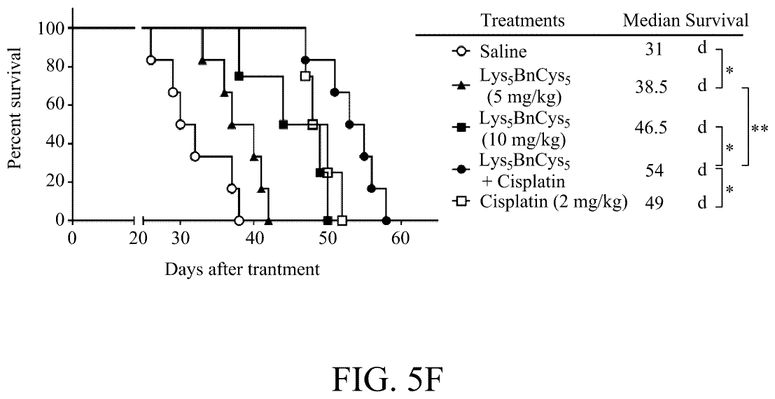

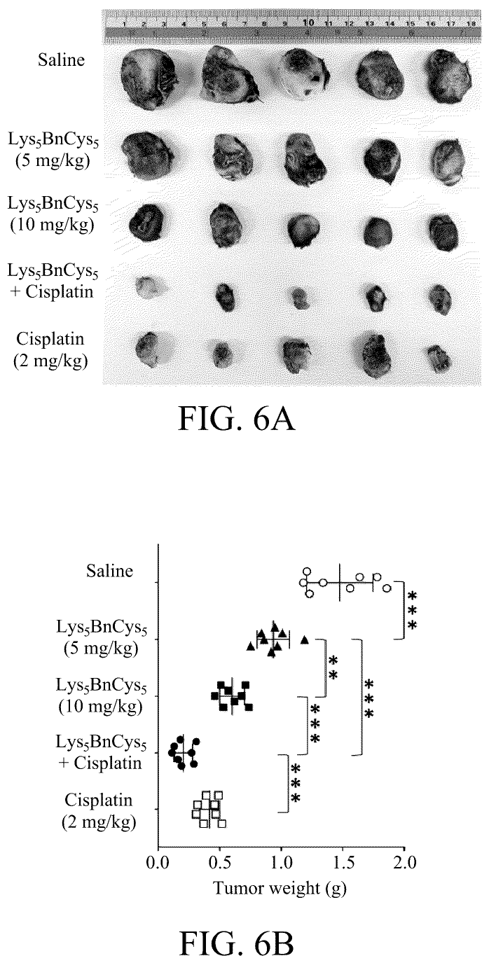

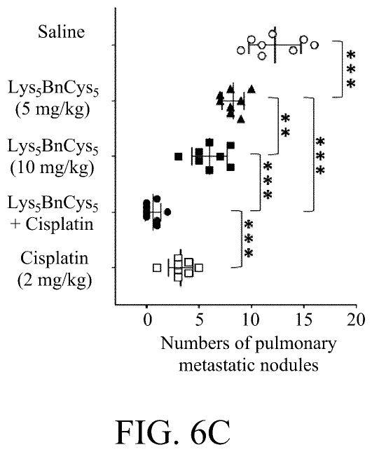

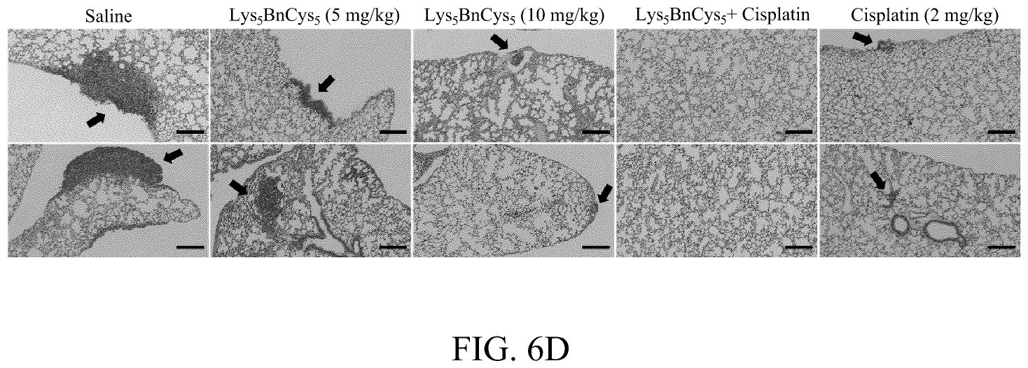

To test possible antitumor effects in Lys₅BnCys₅ (SEQ ID NO: 2), an in vivo syngeneic lung carcinoma model was employed. Briefly, LL2 cells that had been transfected with the luciferase reporter gene were inoculated subcutaneously into C57BL/6 mice. Upon activation of the luciferase gene, luminescence results pointed at less bioluminescence for all experimental copolypeptide groups in comparison to the saline control group (FIGS. 5A-5B), revealing suppression of tumor growth due to Lys₅BnCys₅ (SEQ ID NO: 2) exposure. Cisplatin is commonly used to treat lung carcinoma, yet has been reported side effects such as weight loss (an important indicator of cachexia), forcing patients to withdraw from treatment. In this model of the present disclosure, a combination treatment of Lys₅BnCys₅ (SEQ ID NO: 2) with cisplatin exhibited stronger tumor growth inhibition than the copolypeptide alone (FIGS. 5C-5D). As expected, any group treated with cisplatin exhibited weight loss (FIG. 5E), but mice treated with the peptide alone did not present any significant weight loss, while the group treated with a combination of the peptide and cisplatin exhibited comparable weight loss to cisplatin alone (FIG. 5E). Remarkably, mice treated with the peptide exhibited significantly longer survival time compared to the control group (FIG. 5F). Based on these results, Lys₅BnCys₅ (SEQ ID NO: 2) on tumor metastasis by investigating its ability to inhibit pulmonary metastatic nodules was tested. Said tumors were excised once the saline control group with tumors reached a volume of 1800-2000 mm³ (day 32) after treatment (FIGS. 6A-6B). The peptide-treated tumors exhibited a significantly lower number and size of metastatic nodules (FIGS. 6C-6D). These results reveal that Lys₅BnCys₅ (SEQ ID NO: 2) can effectively suppress tumor growth and extend survival time for tumor-bearing mice. Treatment of Lys₅BnCys₅ (SEQ ID NO: 2) in combination with cisplatin further improved efficacy.

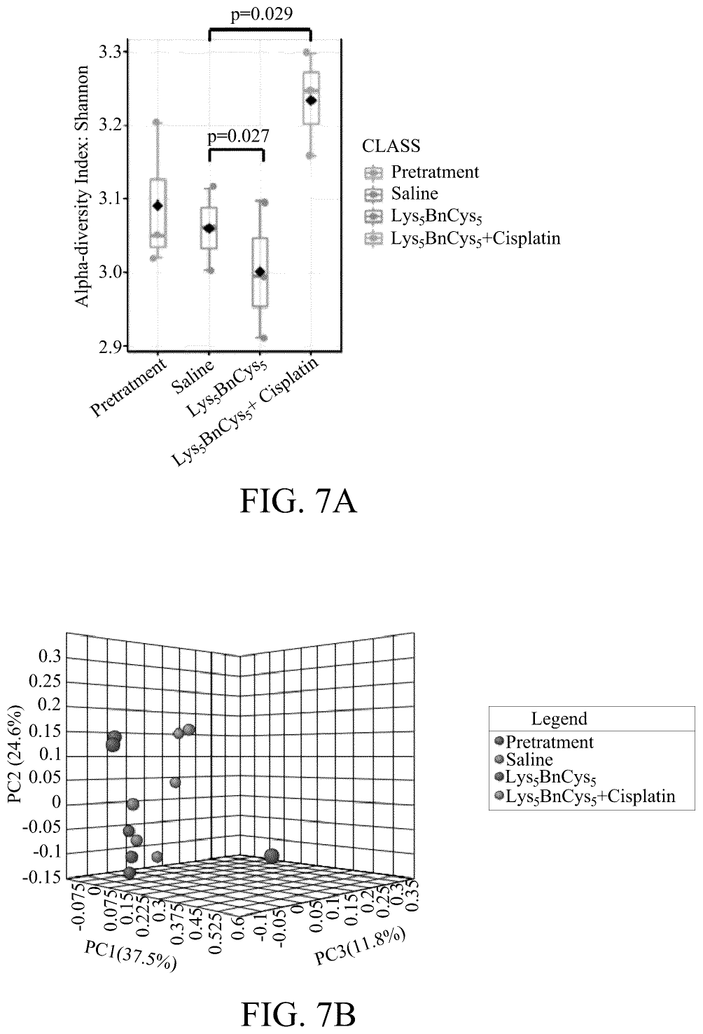

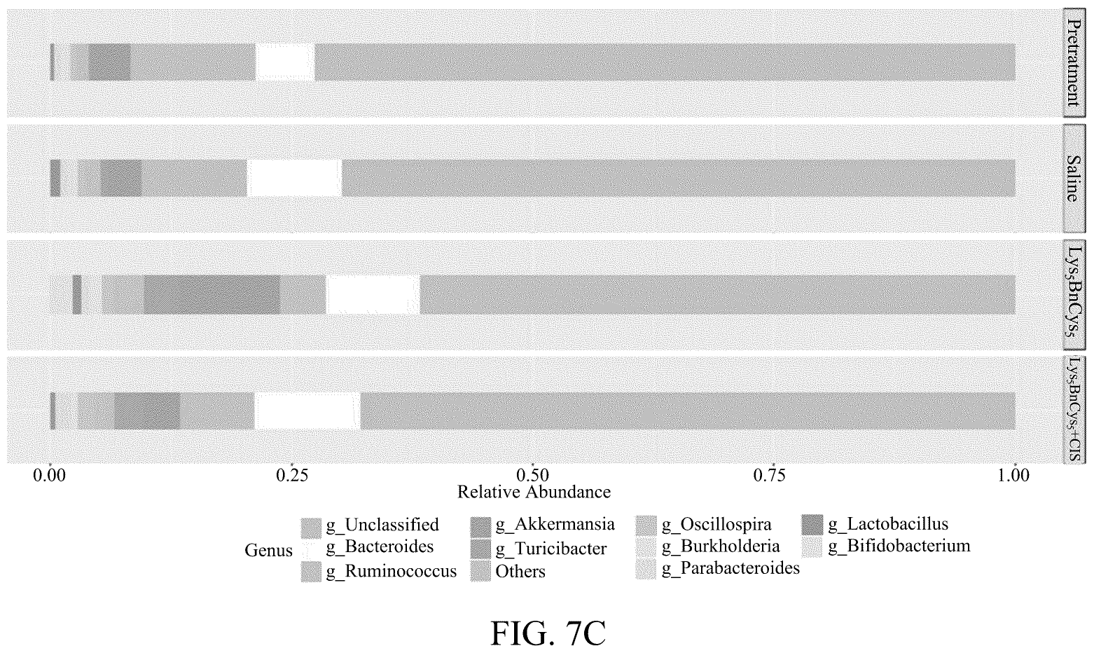

Based on these results, the present disclosure found that lowering the expression of E-cadherin (as seen by immunoblotting) depended on Lys₅BnCys₅ (SEQ ID NO: 2) influencing protein packing, not depended on genetic regulation. As shown in FIGS. 6C-6D, these anti-metastatic in vitro results were consistent with the syngeneic lung carcinoma model of the present disclosure. In addition, Lys₅BnCys₅ (SEQ ID NO: 2) treatment induced caspase-3 dependent apoptotic pathway but did not induce adverse effects, such as weight loss (FIG. 5E). As shown in FIGS. 6A-6D and 7A-7D, Lys₅BnCys₅ (SEQ ID NO: 2) treatment significantly reduced tumor growth and prolongs tumor-bearing mice survival (FIG. 5F), making Lys₅BnCys₅ (SEQ ID NO: 2) treatment as an anticancer peptide.

### Example 5: Lys₅BnCys₅ (SEQ ID NO: 2) Treatment Increased the Relative Proportion of A. muciniphila in the Gut

One having ordinary skills in the art will understand that in some instances, prolonged progression-free survival (PFS) in lung cancer patients with high microbial diversity compared with those of low diversity. To explore whether the composition of gut microbes was altered by Lys₅BnCys₅ (SEQ ID NO: 2) and if the treatment-mediated microbial alterations were potentially linked to its effects on tumor growth, the proportions of 16S rDNA amplicons were analyzed upon each treatment group. Although the Lys₅BnCys₅ (SEQ ID NO: 2) treatment group showed a lower level of α-diversity as compared with saline group based on the Shannon index (both in richness and evenness) (FIG. 7A, P=0.027), its co-treatment with cisplatin significantly increased the microbial α-diversity in the gut (FIG. 7A, P=0.029), suggesting Lys₅BnCys₅ (SEQ ID NO: 2) could provide some survival benefits to patients treated with first-line cisplatin. Further analysis for β-diversity was done with principal coordinate analysis (PCoA) of D_0.5 UniFrac. The PERMANOVA test displayed significant difference in the overall microbial composition between groups (FIG. 7B, P<0.001). The top 10 abundant gut bacteria in Genus levels are illustrated by an OTU table (FIG. 7C). Intriguingly, Ruminococcus spp. was decreased upon Lys₅BnCys₅ (SEQ ID NO: 2) treatment as compared with pre-treatment and saline group (data not shown, P<0.01 and <0.05, respectively). Of note, anti-PD-1 non-responders exhibited a prevalence in Ruminococcus spp.

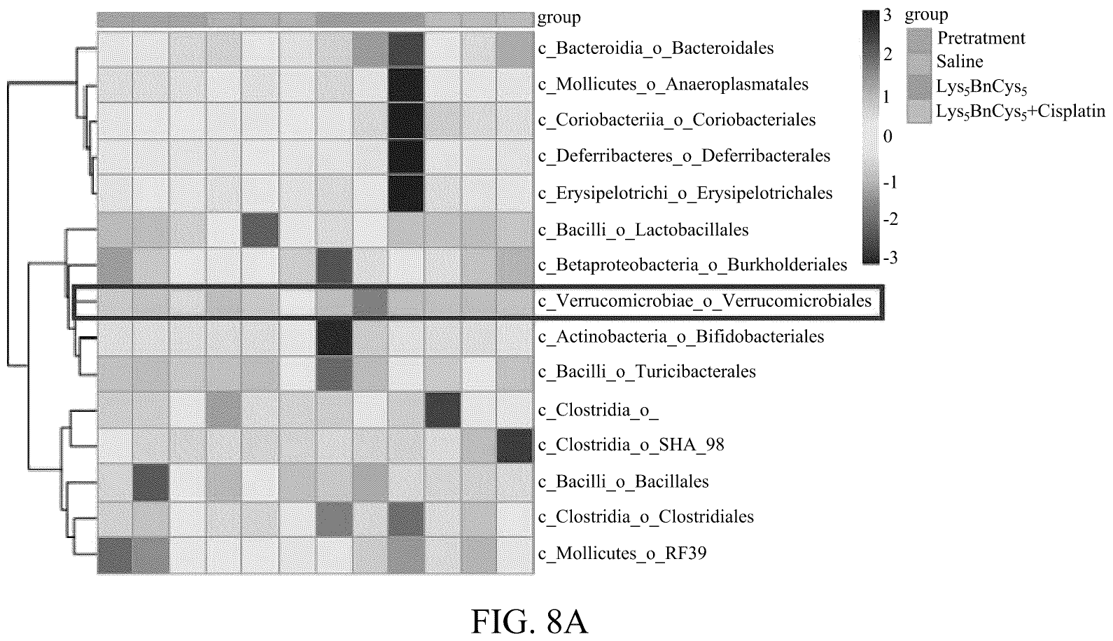

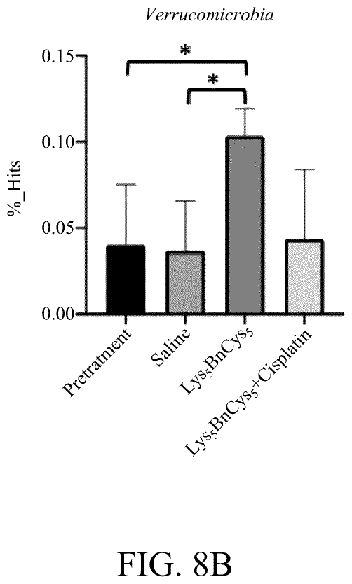

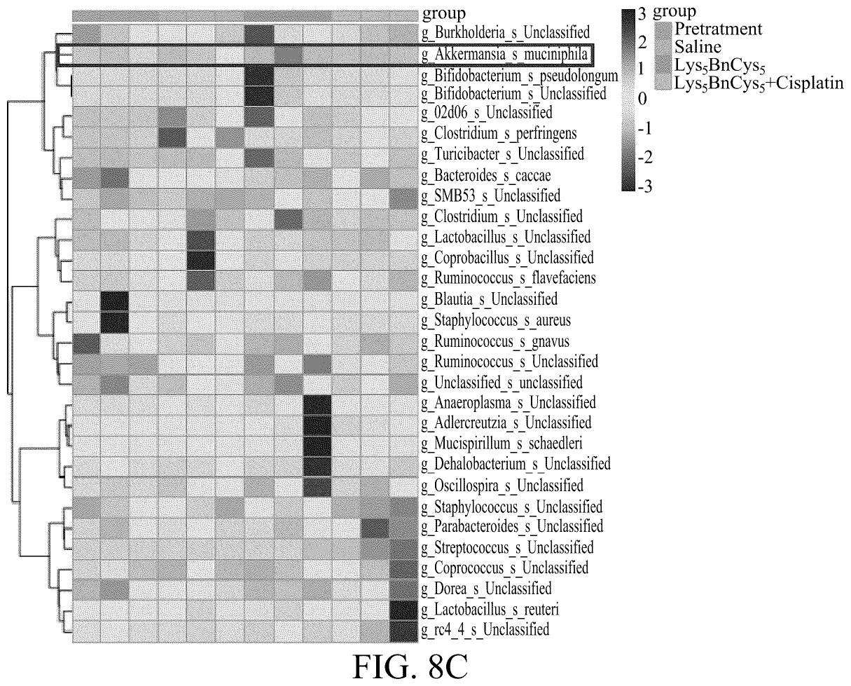

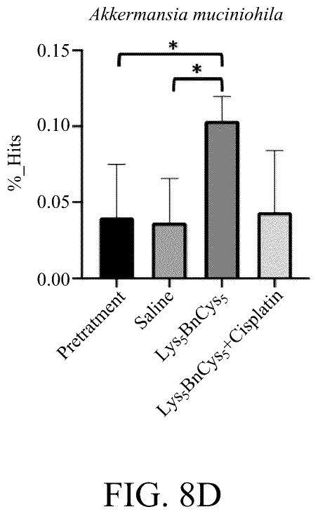

Furthermore, heatmaps showed an abundance of gut microbes in different clusters from each sample among the four subgroups (FIGS. 8A-8D). In phylum levels, Verrucomicrobia was significantly increased by Lys₅BnCys₅ (SEQ ID NO: 2) treatment as compared with pre-treatment and saline group (FIGS. 8A-8B, P<0.05 and <0.05, respectively). Meanwhile, Akkermansia muciniphila (Akk), the only species in the Verrucomicrobia phylum, increased accordingly after Lys₅BnCys₅ (SEQ ID NO: 2) treatment in comparison with pre-treatment and saline groups (FIGS. 8C-8D, P<0.05 and <0.05, respectively), suggesting that one of the mechanisms underlying the impact of Lys₅BnCys₅ (SEQ ID NO: 2) on tumor growth is due to the enrichment of Akk. For example, Akk has been found to enhance the antitumor effect of cisplatin in Lewis lung cancer mice.

One having ordinary skills in the art will understand that in some instances, Akk, one of the most dominant bacteria, exists in the mucus layer of the intestinal tract, constituting 1-4% of the total bacterial cells in healthy adult feces and that Akk may have beneficial effects on its host. For example, differential expression of tumor-associated genes and altered gut microbiome with increased Akk afforded a tumor-preventive microenvironment in intestinal epithelial Pten-deficient mice. The relative abundance of Akk has also been correlated with Immune checkpoint inhibitors (ICIs) through the regulating of the PD-1/PD-L1 axis. ICIs attract sustained clinical responses in a sizable minority of tumor sufferers, achieving significant efficacy in the treatment of advanced lung cancer.

One having ordinary skills in the art will understand that in some instances, intestinal microbiota participates in the body's immune regulation and optimize the therapeutic effects of immune checkpoint inhibitors during cancer progression. Immune checkpoint blockade in cancer immunotherapy also exhibits heterogeneous therapeutic effects in different individuals, partially attributed to gut microbiota. Akk is one of the most dominant bacteria and resides in the mucus layer of the intestine, accounting for 14% of the total number of bacteria in the feces of healthy adults. In the present disclosure, Lys₅BnCys₅ (SEQ ID NO: 2) influenced microbiome composition during treatment. After treatment with Lys₅BnCys₅ (SEQ ID NO: 2) for three weeks, Akk significantly increased in compared to pre-treatment, saline and Lys₅BnCys₅ (SEQ ID NO: 2) mixed cisplatin groups (FIGS. 8A-8D). The anticancer efficacy of Lys₅BnCys₅ (SEQ ID NO: 2) of the present disclosure may be enhanced due to the modulation of intestinal microbiota.

According to the Examples, the copolypeptides of the present disclosure not only affects migration and adhesion of target cells (e.g. cancer cells) but also induces apoptotic activity. These effects may be due to their interfering with E-cadherin expression on the cell membrane and/or intercalating into the cell membrane, triggering caspase-3 and PARP related apoptotic signaling, thus causing DNA condensation. Syngeneic mouse studies of the present disclosure showed that tumor growth and metastasis were effectively inhibited with the treatment of the copolypeptides plus cisplatin without incurring weight loss, prolonging the survival time for tumor-bearing mice. In addition, the copolypeptide therapy of the present disclosure increased the relative proportion of Akk in intestinal microbiota. The synergistic effect of inhibiting E-cadherin (cell migration/adhesion) and modulating intestinal microbiota exhibited by the copolypeptides introduces potent anticancer activity.

### Example 6: Star-Shaped Copolypeptides Inhibit Cancer Cell Migration and Adhesion

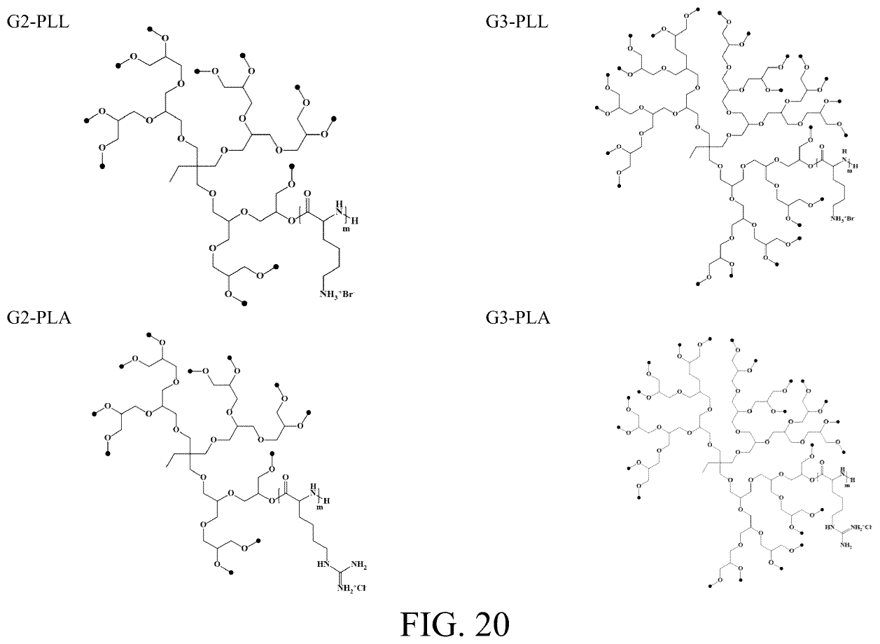

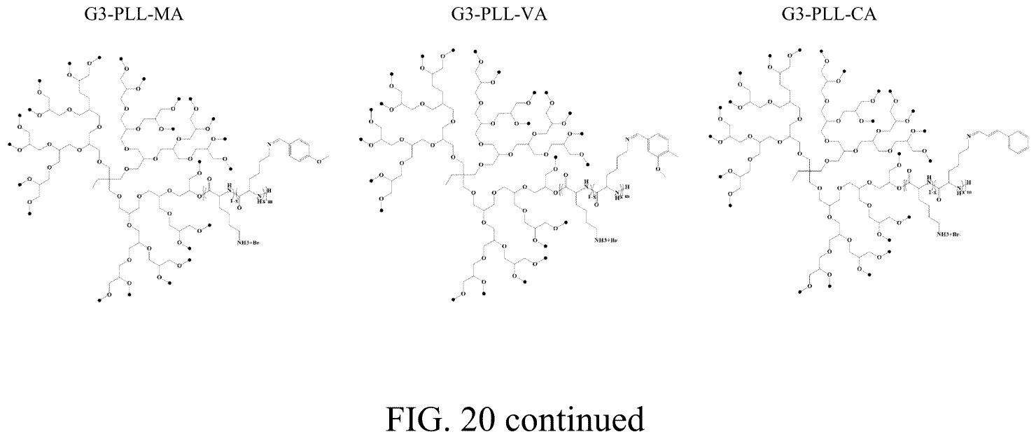

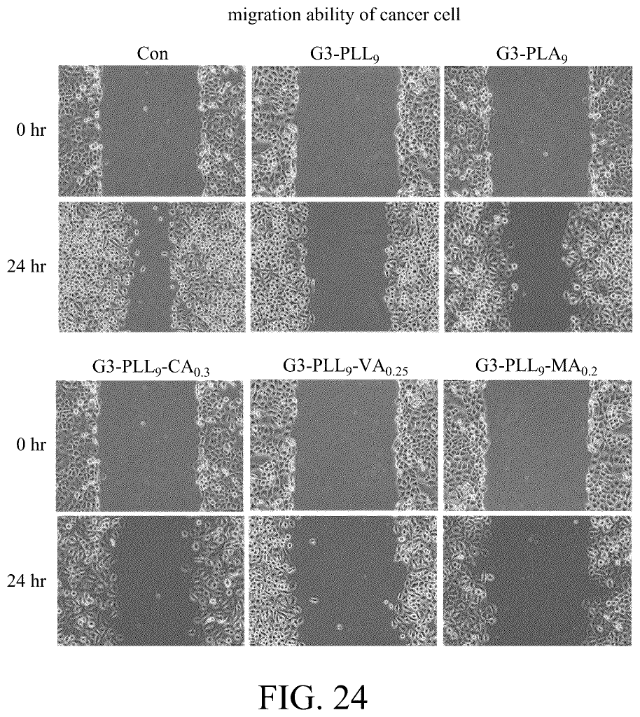

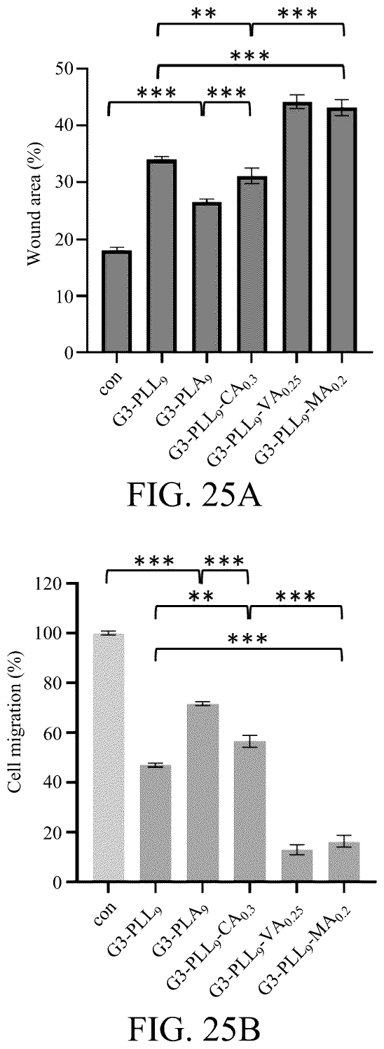

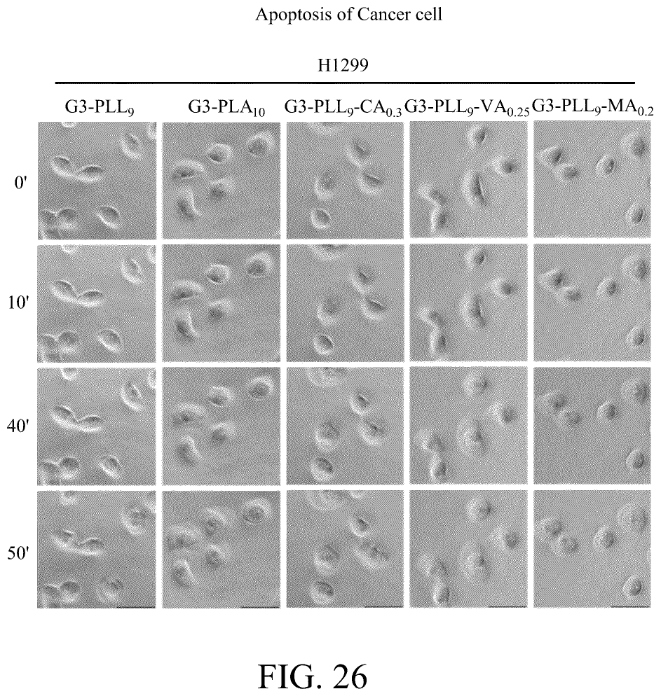

In order to explore more copolypeptides that could affect cell migration, a wound healing assay was performed using similar procedures described above for linear (single-chain) and shorter copolypeptides (i.e. Lys₅BnCys₅ (SEQ ID NO: 2) and Lys₅Cys₅ (SEQ ID NO: 1)). After a scratch line was induced, H1299 cells were treated with copolypeptides G3-PLL₉ (having a side chain of SEQ ID NO: 15), G3-PLA₉ (having a side chain of SEQ ID NO: 16), G3-PLL₉-CA_{0.3}, G3-PLL₉-VA_{0.25}, or G3-PLL₉-MA_{0.2} (their chemical structure are shown in FIG. 20) for 24 h. As shown in FIGS. 24 and 25A-25B, both G3-PLL₉ (having a side chain of SEQ ID NO: 15) and G3-PLA₉ (having a side chain of SEQ ID NO: 16) inhibited cell migration and the addition of low dose cinnamaldehyde (CA, 0.3 μM), vanillin (VA, 0.25 μM), or p-methoxybenzaldehyde (MA, 0.2 μM) to modify side chain of poly-L-Lysine enhanced these effects. Moreover, G3-PLL₉-VA_{0.25} and G3-PLL₉-MA_{0.2} treatment exhibited larger wound healing area than G3-PLA₉ (having a side chain of SEQ ID NO: 16) under the same concentration. The loss of integrity caused by G3-PLL₉-VA_{0.25} and G3-PLL₉-MA_{0.2} decreased cell migration. Thus, the addition of VA or MA group on G3-PLL₉ (having a side chain of SEQ ID NO: 15) seems to play a critical role in suppression of cell migration.

### Example 7: Linear Copolypeptides Enhance Cytotoxicity Against Cancer Cells

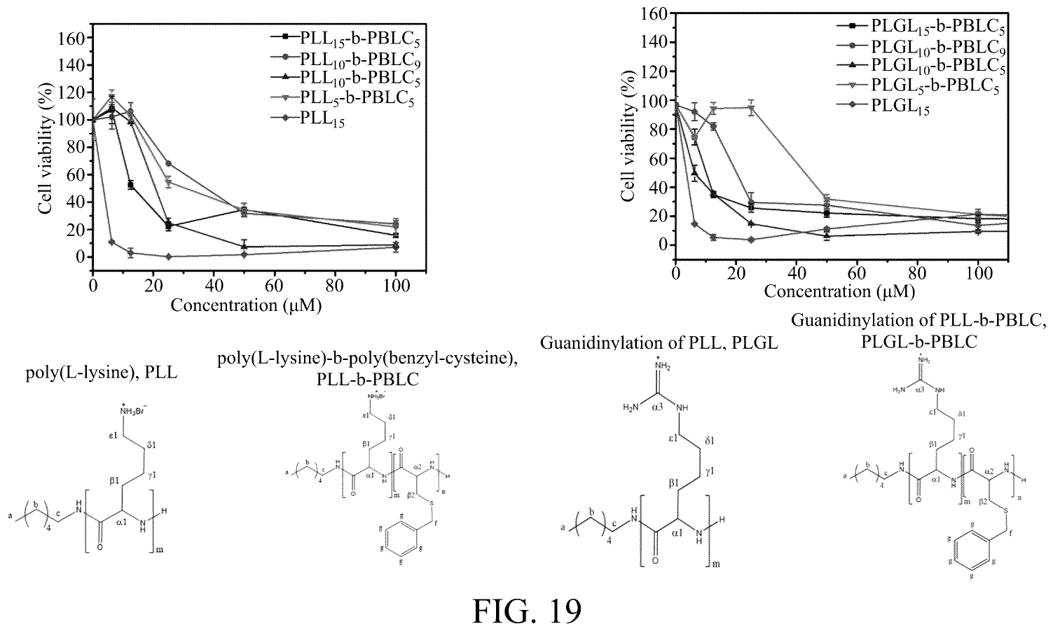

To conform the response to treatment of various linear copolypeptides, the cell viability (%) was determined using similar protocols described above for the linear (single-chain) and shorter copolypeptides (i.e. Lys₅BnCys₅ (SEQ ID NO: 2) and Lys₅Cys₅ (SEQ ID NO: 1)) and the results are shown in FIG. 19. 4 types of linear copolypeptides with various segment lengths: poly-L-lysine (PLL₁₅, SEQ ID NO: 7), poly-L-guanidinylated lysine (PLGL₁₅ (SEQ ID NO: 12)), poly-L-lysine-block-poly-S-benzyl-L-cysteine (PLL-b-PBLC, for example, PLL₁₅-b-PBLC₅ (SEQ ID NO: 3), PLL₁₀-b-PBLC₉ (SEQ ID NO: 4), PLL₁₀-b-PBLC₅ (SEQ ID NO: 5), and PLL₅-b-PBLC₅ (SEQ ID NO: 6)), and poly-L-guanidinylated lysine-block-poly-S-benzyl-L-cysteine (PLGL-b-PBLC, for example, PLGL₁₅-b-PBLC₅ (SEQ ID NO: 8), PLGL₁₀-b-PBLC₉ (SEQ ID NO: 9), PLGL₁₀-b-PBLC₅ (SEQ ID NO: 10), and PLGL₅-b-PBLC₅ (SEQ ID NO: 11)) at concentrations ranging from 0 to 100 μM were used for treating cancer cells (H1299), and the results showed that the cell viability decreased with increasing concentration of linear copolypeptides.

### Example 8: Star-Shaped Copolypeptides Enhance Cytotoxicity Against Cancer Cells

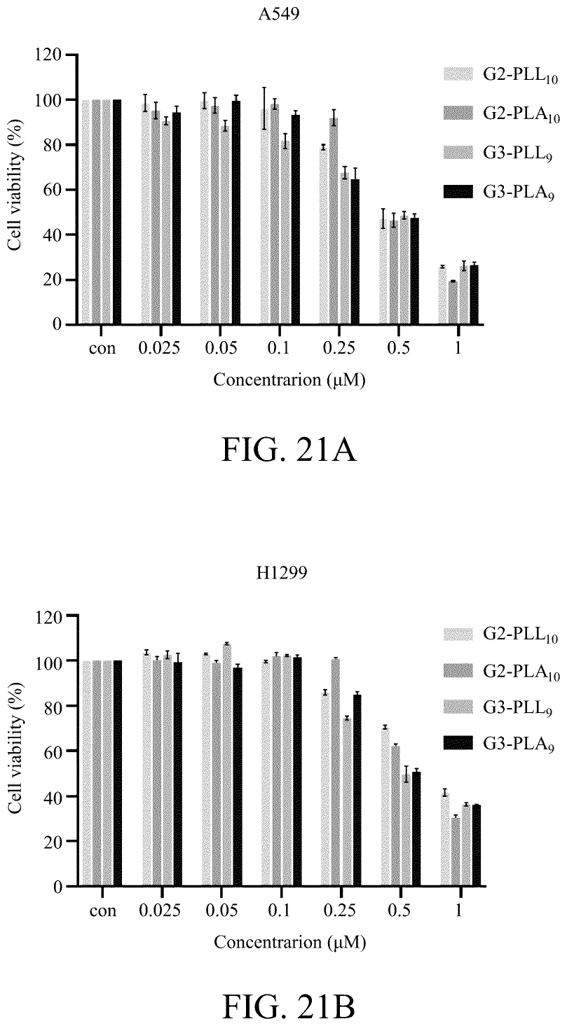

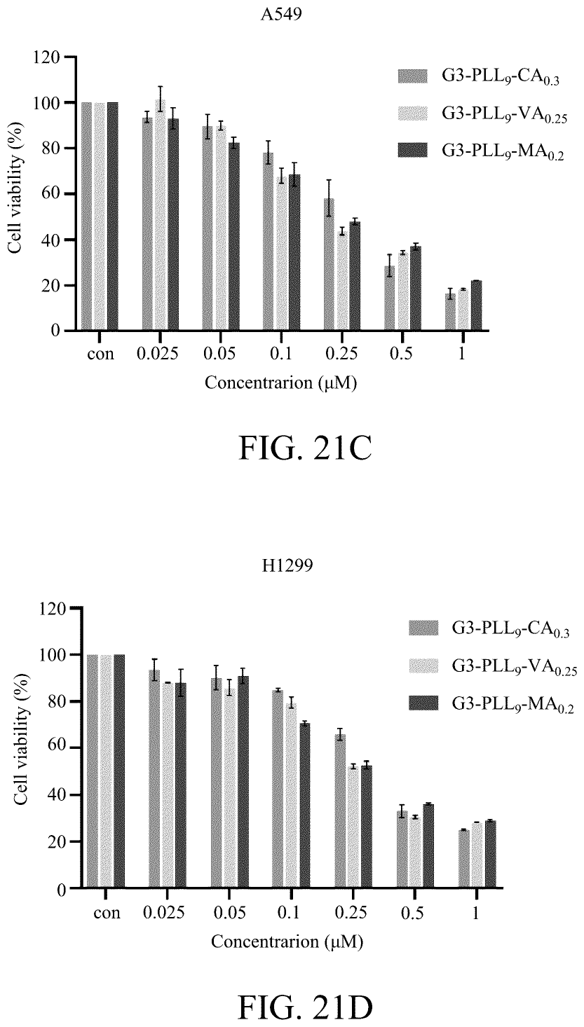

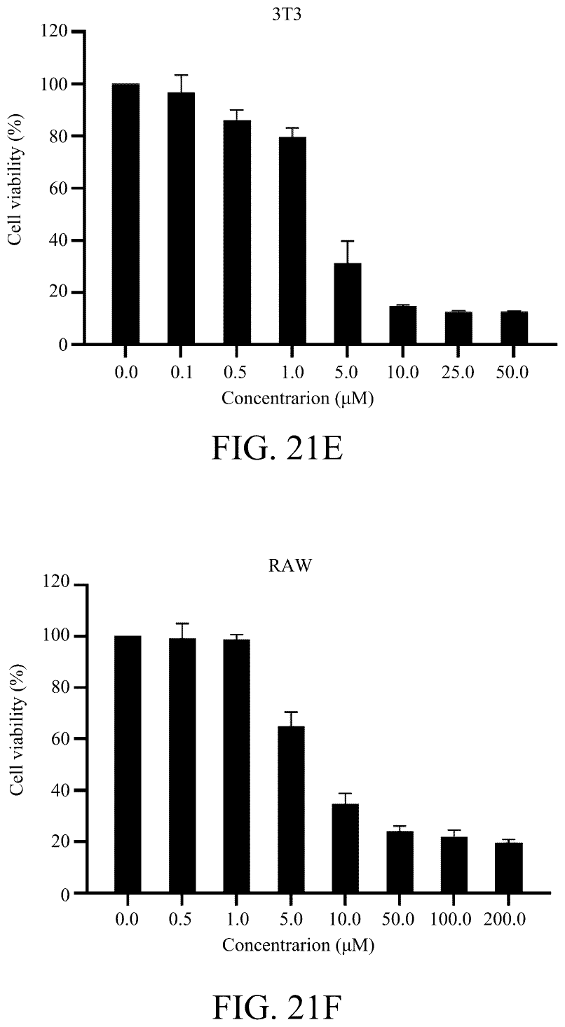

Similar to Example 7, the cell viability (%) was determined using similar protocols described above. In FIGS. 21A-21D, star-shaped copolypeptides: G2-PLL₁₀ (having a side chain of SEQ ID NO: 13), G2-PLA₁₀ (having a side chain of SEQ ID NO: 14), G3-PLL₉ (having a side chain of SEQ ID NO: 15), G3-PLA₉ (having a side chain of SEQ ID NO: 16), G3-PLL₉-CA_{0.3}, G3-PLL₉-VA_{0.25}, or G3-PLL₉-MA_{0.2} (their chemical structure are shown in FIG. 20, wherein G3-PLL₉-CA_{0.3} corresponds to G3-PLL-CA that [1-x]=9 and m=0.3; G3-PLL₉-VA_{0.25} corresponds to G3-PLL-VA that [1-x]=9 and m=0.25; G3-PLL₉-MA_{0.2} corresponds to G3-PLL-MA that [1-x]=9 and m=0.2) at 0.025, 0.05, 0.1 0.25, 0.5 and 1 μM were used for treating A549 and H1299 cells, and the decrease in cell viability became obvious with increasing concentration.

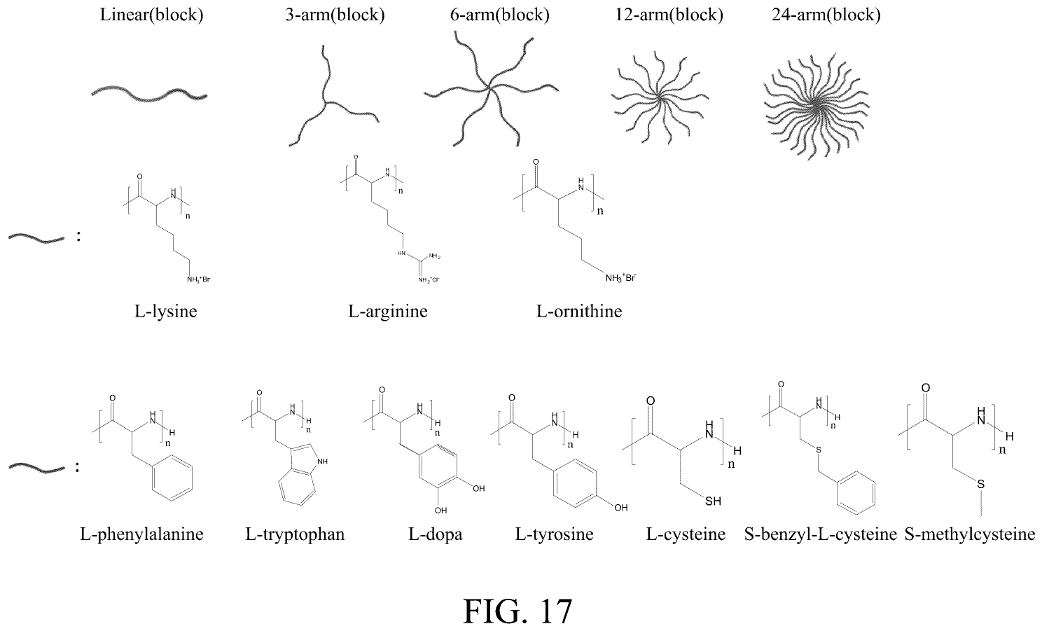

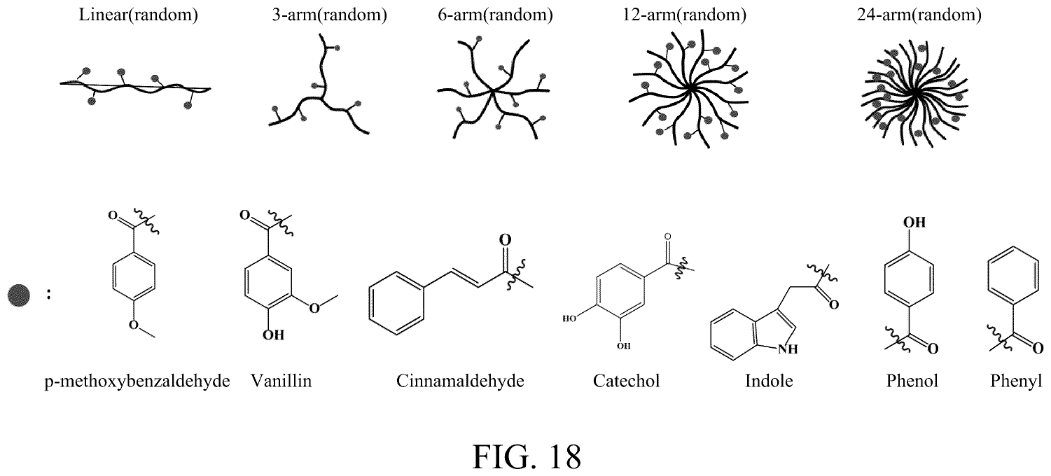

On the other hand, in FIGS. 21E-21F, a star-shaped copolypeptide G3-PLL₁₀-PLF₅ (having a side chain of SEQ ID NO: 17) (i.e., G3-PLL in FIG. 20 that m=10 and further containing poly-L-phenylalanine segment as shown in FIG. 17 that n=5) at 0 to 200 μM was also used for treating 3T3 and RAW cells, and the decrease in cell viability became obvious with increasing concentration.

### Example 9: Star-Shaped Copolypeptides Induce Cancer Cell Apoptosis

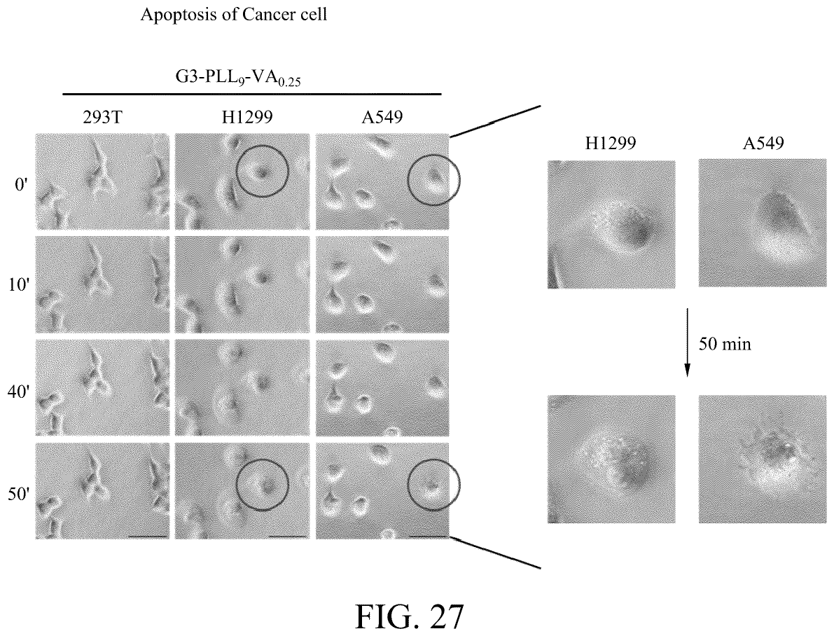

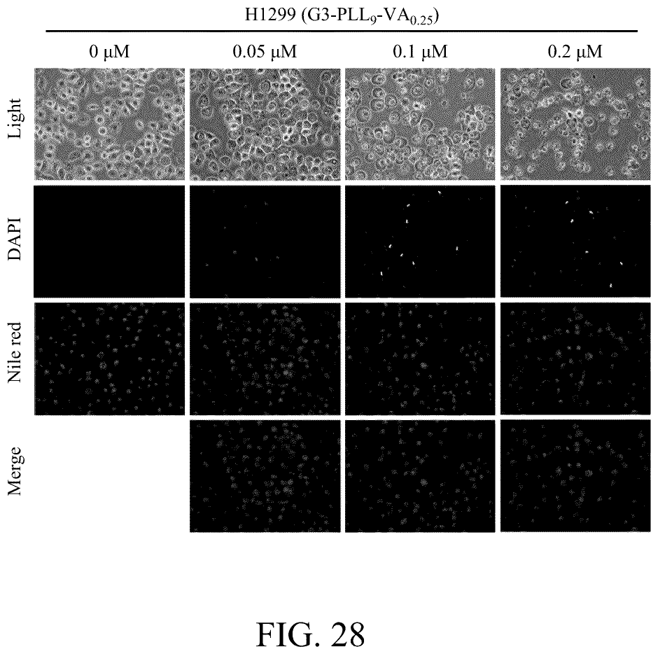

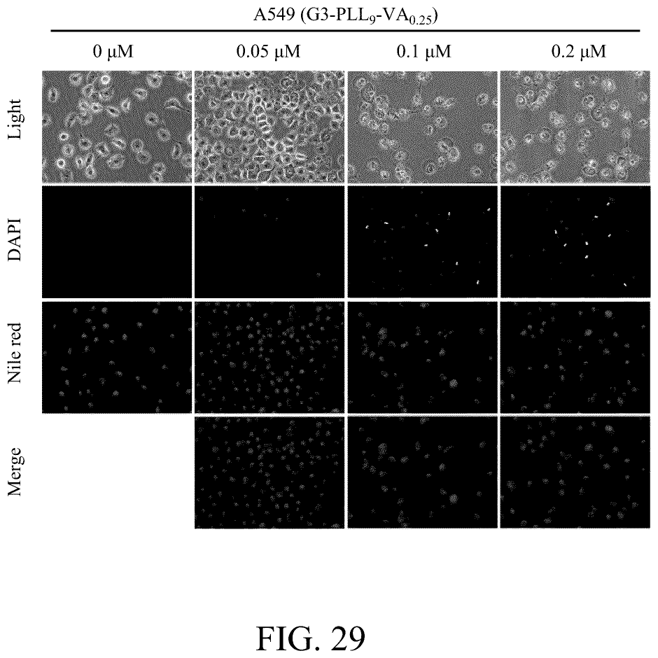

As shown in FIG. 26, after 50′ (min) in G3-PLL₉ (having a side chain of SEQ ID NO: 15), G3-PLA₁₀ (having a side chain of SEQ ID NO: 16), G3-PLL₉-CA_{0.3}, G3-PLL₉-VA_{0.25}, and G3-PLL₉-MA_{0.2} treatment, flatter cell, leakage of intracellular contents, and loss of the original saturation were observed in H1299 cells. In FIGS. 27-29, G3-PLL₉-VA_{0.25} treatment resulted in flatter cell, leakage of intracellular contents, and loss of the original saturation of H1299 and A549 cells, compared with control group (293T cells). In particular, as shown in FIG. 27, these cancer cell structures showed tears and clustering as observed in the images in after about 50 minutes of the G3-PLL₉-VA_{0.25} treatment.

### Example 10: Linear and Star-Shaped Copolypeptides Increase Transfection Efficiency

LysₘBnCys₅ copolypeptides (m=5, 10, and 15, i.e., SEQ ID NOs: 2, 5, and 3)) efficiently carried p53 gene (pcDNA3 p53 WT plasmid) into H1299 (p53-deficient) cells and enhanced chemotherapeutic treatment. The positively charged polyplexes formed by LysₘBnCys₅ and plasmid DNA exhibited improved transfection efficiency which increased with increments of the PLL to PBLC block ratio. Improved transfection efficiency was mainly attributed to optimal polyplex stability, determined by peptide charge density and rigidity. In particular, the transfection rate using Lys₁₅BnCys₅ (SEQ ID NO: 3) as gene vectors was almost equivalent to that using lipofectamine 2000. The in vitro tests showed that the LysₘBnCys₅ gene vector reversed the cisplatin sensitivity of p53-null cancer cells by increasing apoptotic signaling. Further, xenograft mouse studies revealed that LysₘBnCys₅/p53 gene therapy significantly suppressed tumor growth and enhanced low-dose cisplatin treatment. Moreover, treatment with both LysₘBnCys₅/p53 gene vector and cisplatin extended the survival rates of tumor-bearing mice.

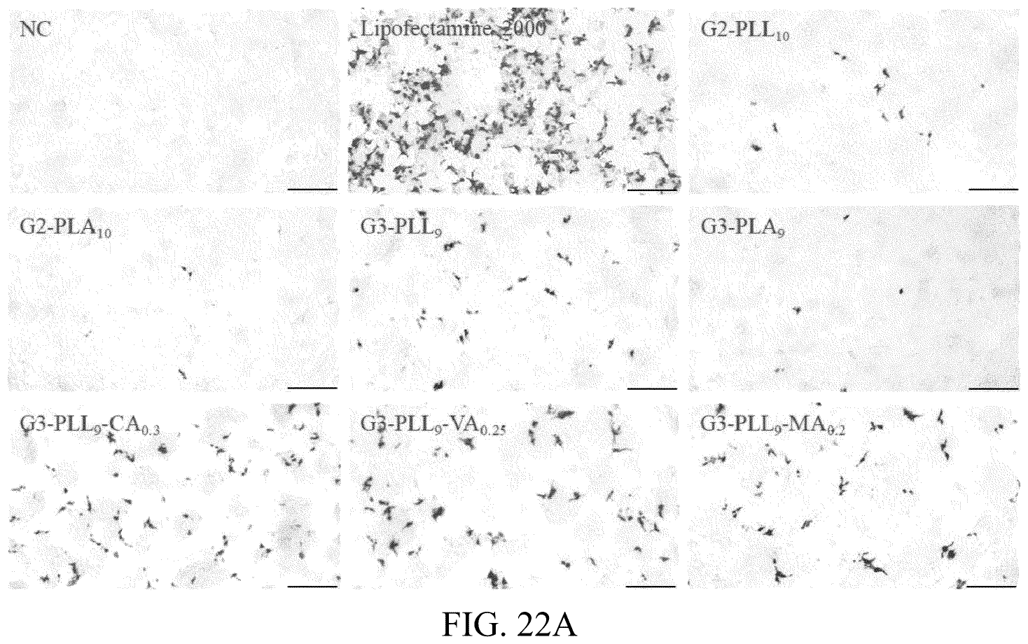

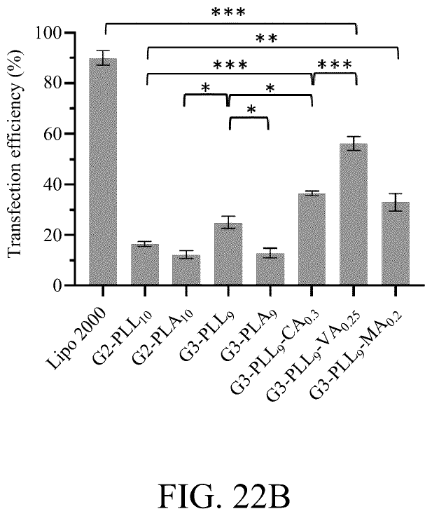

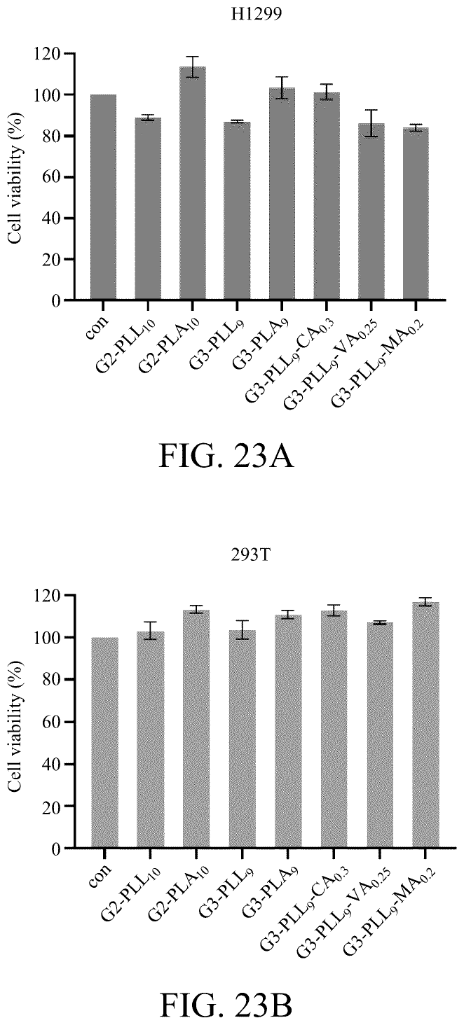

FIGS. 22A-22B are results showing the p53 gene transfection efficiencies of various star-shaped copolypeptides to H1299 cells. Among various star-shaped copolypeptides, G3-PLL₉-VA_{0.25} showed a better transfection efficiency more than half of transfection efficiency of lipofectamine 2000. The results of cell viability (H1299 and 293T cells) response to treatment of star-shaped copolypeptides/pDNA complexes shown in FIGS. 23A-23B revealed that normal cells (293T) were not damaged in the treatment.

### Material and Methods

Linear Copolypeptide Synthesis (LysₘBnCysₙ and LysₘCysₙ)

L-lysine was reacted with benzyl chloroformate to prepare Z-L-lysine (Z-Lys). Z-Lys and S-Benzyl-L-cysteine (BnCys) are reacted to form N-carboxy anhydrides(NCAs): Z-Lys-NCAs and BnCys-NCAs, respectively. Z-LysₘBnCysₙ copolypeptide was synthesized by the ring-opening polymerization (ROP) from Z-Lys-NCAs and BnCys-NCAs using hexylamine as the initiator, and the LysₘBnCysₙ copolypeptide was then obtained by deprotection of the Z group (e.g. using hydrogen bromide HBr).

For the synthesis of the LysₘCysₙ copolypeptide, Z-LysₘBnCysₙ copolypeptide was dissolved in trifluoroacetic acid (TFA, Sigma-Aldrich) in a round bottom flask covered with aluminum foil to avoid light. 33 wt % of hydrogen bromide solution (Sigma-Aldrich) was added to the solution and the mixture was stirred for 30 minutes. Subsequently, the mixture was stirred for 2 more hours upon the addition of trimethylsilyl iodide (TMSI, Alfa Aesar). The crude product was obtained by pouring the mixture into excess ether (>99%, ECHO), centrifuging, and then dissolving it in deionized (DI) water. Upon adjusting the solution pH to a neutral condition, the resulting solution was dialyzed against DI water for 48 hours using a 1 kD dialysis tube. Finally, the LysₘCysₙ peptide was obtained through lyophilization.

### Instrumentation and Characterization of Linear Copolypeptide Synthesis

Lys₅BnCys₅ (SEQ ID NO: 2) and Lys₅Cys₅ (SEQ ID NO: 1) peptides dissolved in DMSO-d6 as well as Z-Lys₅ and Z-Lys₅BnCys₅ peptides dissolved in TFA-d₁ were analyzed by a BRUKER ADVANCE III HD NMR (600 MHz). The MALDI-TOF MS spectrum of the side-chain protected peptide was recorded in the reflection mode of a MALDI-TOF spectrometer (Bruker Autoflex III TOF/TOF). The secondary conformations of peptides were characterized by Fourier transform Infrared (FTIR) and circular dichroism (CD) analyses on a Thermo Nicolet 6700 FTIR spectrometer and JASCO J-815 spectrometer, respectively. For CD measurements, the concentration of peptide samples in DI water was 0.1 mg mL⁻¹. SAXS patterns of peptide solutions were recorded on a Bruker NanoSTAR U diffractometer with controlled voltage 45 kV and current 650 μA at room temperature under 4×10⁻¹ torr. The critical aggregation concentration (cac) of peptides was determined by measuring the pyrene emission spectra of various peptide solutions with an integration time of 1.0 sec using a Hitachi FL-4500 fluorescence spectrophotometer. Peptide solutions with concentrations ranging from 5×10⁻³ to 0.5 mg mL⁻¹ were prepared for the measurements (Huang et al. 2011, and Chen et al. 2015)

### Synthesis of Star-Shaped Copolypeptides (3 Armed or 6 Armed Lysₘ-Cysₙ or Lysₘ-BnCysₙ)

The star-shaped copolypeptides with 3 arms or 6 arms (for example, 3 armed or 6 armed Lysₘ-Cysₙ or Lysₘ-BnCysₙ) were synthesized by using the respective polyol initiators, which were 1,1,1-tris(hydroxymethyl)propane (THMP, 98%, Aldrich) and dipentaerythritol (DPET, 90%, Alfa Aesar), promoted by 1,1,3,3-tetramethylguanidine (TMG, 99%, Aldrich). For the synthesis of 3 armed Z-Lys₁₅-BnCys₅, a Z-Lys-NCA (1.0 M in anhydrous dimethylformamide (DMF)) solution and a THMP (0.02 mM in anhydrous DMF) solution were prepared in a glove box. Then a TMG stock solution (54.5 mM in DMF) was prepared and added to the THMP solution, reaching a final concentration of 6.0 mM. Upon adding a designated amount of the solution to the Z-Lys-NCA solution, the reaction mixture was stirred at 30° C. for 72 h under an argon atmosphere. A designated amount of the BnCys-NCA (1.0 M in anhydrous DMF) was added to the reaction mixture and stirred at 30° C. for additional 72 h. The final molar ratio of THMP, Z-Lys-NCA and BnCys-NCA was set to be 1:60: 15. Finally, a white solid was obtained by lyophilization of the reaction mixture after dialyzing against methanol and deionized (DI) water for 48 and 24 h, respectively. The typical yields for the linear and star-shaped diblock copolypeptides were between 80 and 90%.

For the synthesis of 3 armed Lys₁₅BnCys₅ copolypeptide (having a side chain of SEQ ID NO: 3), 3 armed Z-LysₘBnCysₙ copolypeptide was first dissolved in trifluoroacetic acid (TFA, Sigma-Aldrich) in a round bottom flask covered with aluminum foil to avoid light and 33 wt % of hydrogen bromide solution (Sigma-Aldrich) was added to the solution. The mixture was stirred for 30 minutes, followed by precipitation using ethyl ether (>99%, ECHO) and lyophilization after dialysis against DI water (yield: 90 to 95%).

### Characterization of Star-Shaped Copolypeptides

The star-shaped copolypeptides were characterized by NMR, gel permeation chromatograph-light scattering (GPC-LS), and MALDI-TOF MS. The results of NMR were obtained by using 2D 1H-13C HSQC NMR. The GPC-LS system equipped with three Viscotek detectors and two Shodex GPC columns was operated at 55° C. and 0.8 mL/min of flow rate. The eluent was DMF containing 0.1 M LiBr and polystyrene (molecular weight: 25000 g/mol) was used as the calculation standard. The samples were dissolved in DMF and passed through a PTFE filter (0.2 μm, 13 mm, Finetech) before GPC analysis. The MALDI-TOF MS spectra were recorded in reflection mode of a MALDI-TOF spectrometry (Bruker Autoflex III TOF/TOF).

### Cytotoxicity and Hemolysis Assay

The cytotoxicity of copolypeptides at various concentrations or in combination with cisplatin (5 μM) was evaluated by incubating with A549, BEAS-2B, and H1299 cells (5×10³ cells/well) in 96-well plates for 24 h using 10% FBS DMEM as the medium. After adding CCK-8 reagent (Targetmol) dissolved in serum-free medium, the well plates were incubated at 37° C. for 1 h. Upon transferring the medium to fresh 96-well plates, the absorbance of each well was measured at 450 nm using a Microplate Spectrophotometer (BioTek). The percentages of viable cells were calculated by the below equation:

Cell viability (%)=(Absorbance_{test cell}−Absorbance_{blank})/(Absorbance_{control cell}−Absorbance_{blank})×100

The hemolytic activities of copolypeptides were evaluated by treating with human red blood cells (hRBCs). The hRBCs purified by centrifugation, washing with phosphate-buffered saline (PBS; pH 7.0, 35 mM phosphate, 150 mM NaCl) and re-suspending to 10% (v/v) in PBS. The procedure was repeated three times. In sterile 96-well plates, the copolypeptide solutions (100 μL) with designated concentrations were added to the hRBC suspensions (100 μL) and then the well plates were incubated at 37° C. for 1 h, followed by centrifugation at 1500×g for 5 min. Upon transferring the supernatant to fresh 96-well plates, the absorbance (Abs) of the supernatant at 405 nm was measured using a microplate reader for calculating the hemoglobin release. The absorbance (Abs) values of PBS and 0.10% Triton X-100 were measured to define 0% and 100% of hemolysis, respectively. The percentage of hemolysis was calculated as [(Abs_{peptide}−Abs_{PBS})/(Abs_{0.1% Tritonx-100}−Abs_{PBS})]×100.

### Wound-Healing Assay

A wound-healing assay was performed to evaluate the effect of copolypeptides on the migration ability of H1299 cells. Upon seeding cells (1×10⁶ cells/well) into 6-well plates, a 200 μL pipette tip was used to scratch the cellular monolayer, followed by washing with PBS thrice. A fresh medium (10% FBS DMEM) with or without copolypeptides (4 μM or 8 μM) and in combination treatment with cisplatin (5 μM) was then added to the wells with scratched cells. An inverted microscope was used to take photographs after 24 h incubation and the ImageJ software was used to quantify the wound-healing area. The percentage of the wound-healing area was determined on the basis of the total cell area (100%).

### Immunoblot Analysis

The immunoblot analysis was conducted by treating H1299 cells with copolypeptides (4 μM) in the presence or absence of cisplatin (5 μM) for 24 h, followed by extraction using RIPA Lysis and Extraction Buffer (Thermo Fisher Scientific) and then separation by using 8% SDS-PAGE. Upon transferring onto NC membranes (0.22 μm) blocked in 5% fat-free dry milk, the proteins were incubated with primary antibodies against E-cadherin, caspase-3, poly(ADP-ribose) polymerase (PARP), and β-actin, followed by incubation with their appropriate secondary antibodies. An Immobilon Western Chemiluminescent HRP Substrate kit (Merck) was used to form visualized protein bands and the intensities of the protein bands were quantified using the Image J software (Image Processing and Analysis in Java) by normalizing to that of β-actin.

### Immunohistochemistry Analysis

The immunohistochemistry analysis was conducted by treating H1299 cells with copolypeptides (4 μM) in the presence or absence of cisplatin (5 μM) for 20 h. The treated cells were fixed with cold methanol for 10 min and blocked endogenous peroxidase by treating with H₂O₂ solution (3%) in PBS for 30 min, followed by incubating with Triton X-100 (0.3%) in milk diluent/blocking solution (10%, SeraCare) for 1 h and washing with PBS twice. Then the samples were then incubated with mouse anti-E-cadherin (Proteintech) or isotype control IgG (Santa Cruz) at 4° C. overnight, followed by washing with PBS thrice. Finally, the samples were incubated with horseradish peroxidase (HRP)-conjugated goat anti-mouse IgG (Jackson ImmunoResearch) for 2 h. The reactivity was visualized with 3,3′-diaminobenzidine (DAB, brown color, Dako) and counterstained with hematoxylin.

### Solid-Phase Adhesion Assay

The solid-phase adhesion analysis was conducted by treating H1299 (2×10⁵ cells/well) cells seeded into a 12-well plate with or without copolypeptides (4 μM) in the presence or absence of cisplatin (5 μM) for 6 h. Then the cells were detached from culture plates by trypsinization, washed, and resuspended in the serum-free medium. The suspended cells (1×10⁴ cells/well) were seeded into 96-well plates precoated with 2.0 μg/mL fibronectin (Sigma), followed by incubating the well plates at 37° C. for 2 h. The adhered cells were then measured using a CCK-8 assay after removing non-adherent cells by immersing the plates in PBS containing MgCl₂ (1.0 mM). The Abs values of the wells, corresponding to the proportion of cells adhered to the coated wells, were measured at 450 nm using a microplate reader. Experiments were done in quadruplicate and repeated thrice. The percentage of adhesion rate was calculated using the equation (Absₜₑₛₜ/Abs_{control})×100).

### Murine Lung Tumor Model and Luciferase-Based Noninvasive Bioluminescence Imaging

LL2 cells (5×10⁵) expressing a luciferase reporter were inoculated subcutaneously into the backs of C57BL/6 mice, as a lung cancer animal model. After 7 days, saline control or copolypeptides were injected intratumorally to start the beginning of the timeline (day zero). Mice were intratumorally injected (120-150 μL per mouse based on the body weight) with either 2 mg/kg cisplatin, 5 mg/kg Lys₅BnCys₅ (SEQ ID NO: 2), 10 mg/kg Lys₅BnCys₅, 5 mg/kg Lys₅BnCys₅₊₂ mg/kg cisplatin, or saline every other day for 24 days (n=10-12). Tumor growth, body weight, and survival were closely monitored. Tumor volume was calculated using the following equation: (length of tumor)×(width of tumor)²×0.45 by measuring the tumors in two perpendicular axes with a tissue caliper. Once the tumor volume reached 2,000 mm³, the mice were sacrificed and subsequently recorded as dead. The experimental protocol adhered to the rules of the Animal Protection Act of Taiwan and was approved by the Laboratory Animal Care and Use Committee of the National Cheng Kung University. On day 12, mice were intraperitoneally injected with D-luciferin potassium salt (200 μL, 10 mg/mL, Thermo Fisher Scientific), followed by anesthesia with 2% isoflurane in order to perform in vivo bioluminescence imaging. An IVIS-200 Perkin Elmer system with its integrated acquisition and analysis software (Living Image V. 2.50) was performed to quantify the said signals. On day 32, an optical microscope was used to examine the number of pulmonary metastatic nodules.

### Preparation and Characterization of Copolypeptides DNA Polyplexes

Copolypeptides/DNA polyplexes were prepared as follows. Briefly, the copolypeptides and plasmid DNA were separately dissolved in de-ionized (DI) water to prepare homogeneous solutions. The final concentrations of copolypeptides and plasmid DNA (or pEGFP-C1) were 5.0 μM and 4.0 μg mL⁻¹, respectively. Theoretical molecular weight values for PLL₅-b-PBLC₅ (SEQ ID NO: 6), PLL₁₀-b-PBLC₅ (SEQ ID NO: 5), and PLL₁₅-b-PBLC₅ (SEQ ID NO: 3) copolypeptides used for calculation were 2127, 3172, and 4177 g mol⁻¹, respectively. The copolypeptides/DNA polyplexes with different N/P ratios were then prepared by mixing copolypeptide solutions with a designated volume with the DNA (pcDNA3 p53 WT) solution. Mixtures were then vortexed for 30 sec. The sizes of copolypeptides/DNA polyplexes were characterized using a Hitachi H-7500 Transmission Electron Microscope (TEM). The gel electrophoresis analysis of copolypeptides/DNA polyplexes with various N/P ratios was incubated at 25° C. for 30 min, and performed on 0.8% agarose gels (100 V, 30 min).

### Self-Assembled Structure of Copolypeptides

The self-assembled structures of copolypeptides were characterized by a NANOSTAR U SYSTEM Small-Angle X-Ray Scattering (SAXS) equipped with a Vantec-2000 detector and an IμS X-ray source. SAXS patterns were generated by plotting the scattering intensity (I(q)) versus the scattering vectors (q). Copolypeptides and polyplexes in DI water were loaded in quartz capillary tubes for SAXS analysis. The copolypeptide solutions with 1.0 mg mL⁻¹ of copolypeptide concentration were prepared for SAXS analysis. The N/P ratio of the polyplexes was 5 for all PLL-b-PBLC copolypeptides. The voltage and current of the SAXS measurements were set at 45 kV and 0.65 mA, respectively.

### Transfection Efficiency of Copolypeptides/pDNA Polyplexes

5.0 μM copolypeptides or 4.0 μL lipofectamine 2000 (Thermo Fisher Scientific) were mixed with 4.0 μg of plasmid DNA (pEGFP-C, Clontech) in 1 mL serum-free DMEM and left standing for 20 min. After cell concentration reached 2×10⁵ cells/well in 6-well plates, the culture medium was replaced with 1 mL copolypeptides/DNA complexes (polyplexes) and incubated for 6 h. 2% FBS DMEM was then added for a further 24-h incubation period. 10% FBS DMEM was then replaced to treat cells for another 24 h. All the cells were cultured at 37° C. in a humidified atmosphere of 5% CO₂. Green fluorescent protein (GFP) expression of transfected cells was examined under an inverted fluorescence microscope (IX71, Olympus). Quantitative evaluation of the transfection efficiency was also conducted using a flow cytometer (BD Biosciences). The transfected cells were washed with phosphate-buffered saline (PBS) solution and detached with 0.2 mL of 0.1% trypsin-EDTA. A total of 1 mL from each well was collected and then settled with centrifugation. After discarding the liquid phase, the cells were resuspended in 1 mL of PBS. Transfection efficiency was then calculated as the percentage of cells expressing GFP, with the fluorescence of 2×10⁴ cells evaluated by flow cytometry.

Anticancer Effects of p53 Gene Transfection with Chemotherapeutic Drugs

H1299 cells (2×10⁵ cells/well) cultured in 6-well plates were transfected with pcDNA3 p53 WT (1.0-4.0 μg, Addgene) plasmids using copolypeptides (5.0 μM) Bioinformatics Analysis

The raw paired-end reads were trimmed and passed through quality filters (quality trimming, discarding short read length and removing chimeras) and were assigned to operational taxonomic units (OTUs) which shared ≥97% similarity with the Greengene database. The raw paired-end reads were also analyzed with the base space Ribosomal Database Project (RDP) classifier. Operational taxonomic units (relative abundance, heatmap, Krona, and differential abundance analysis), α-diversity (Shannon index), and 0-diversity (PCoA-Unweighted UniFrac) were determined with base space (Illumina), CLC Microbial Genomics Module (Qiagen) and Graphpad Prism 7. The OTU table was generated by CLC Microbial Genomics Module to be further analyzed with linear discriminant analysis effect size (LEfSe) and Phylogenetic Investigation of Communities by Reconstruction of Unobserved States (PICRUSt) analysis. LEfSe was conducted by Galaxy/HutLab to identify specific microbial markers between groups, with an alpha value for the factorial Kruskal-Wallis test/pairwise Wilcoxon test of 0.05 and an LDA score cut-off of 2.0. PICRUSt prediction was conducted by Galaxy/HutLab according to the Kyoto Encyclopedia of Genes and Genomes (KEGG) functional pathways database and analyzed with the Statistical Analysis of Metagenomic Profiles (STAMP) software. The STAMP criteria were set up, removing unclassified reads, with p<0.01 and an effect size of 1. The results identified functional pathways with a significantly different abundance at level 3 between groups.

### Statistical Analysis

The data are reported as the means±standard error of the mean (SEM) with n=3, unless otherwise noted. The data were analyzed using two-way ANOVA and Unpaired Student's t test. Not significant (ns) indicated a statistically insignificant, whereas p<0.05 indicated a statistically significant test, which was set to *p<0.05, **p<0.01, and ***p<0.001.

The above-described descriptions of the detailed embodiments are to illustrate the preferred implementation according to the present disclosure, and it is not to limit the scope of the present disclosure. Accordingly, all modifications and variations completed by those with ordinary skill in the art should fall within the scope of the present disclosure defined by the appended claims.

### REFERENCES

The following references, to the extent that they provide exemplary procedural or other details supplementary to those set forth herein, are specifically incorporated herein by reference.
Y. C. Huang, M. Arham, J. S. Jan, Soft Matter 2011, 7 (8), 3975.
B. Y. Chen, Y. C. Huang, J. S. Jan, RSC Adv 2015, 5 (29), 22783.
Spatola, (1983) in Chemistry and Biochemistry of Amino Acids, Peptides, and Proteins, Weinstein, ed., Marcel Dekker, New York, p. 267.

What is claimed is:

1. A block copolypeptide, comprising:
a first positively charged peptide segment consisting of 5 to 20 substituted or unsubstituted amino acids selected from the group consisting of L-lysine, L-arginine, L-omithine, and L-homoarginine.

2. The block copolypeptide of claim 1, being a linear or branched block copolypeptide comprising a second hydrophobic peptide segment consisting of 5 to 20 substituted or unsubstituted amino acids selected from the group consisting of L-phenylalanine, L-tryptophan, L-dopa, L-tyrosine, L-cysteine, S-benzyl-L-cysteine, and S-methyl cysteine.

3. The block copolypeptide of claim 1, being a star-shaped block copolypeptide comprising a polyol initiator as a core.

4. The block copolypeptide of claim 3, further comprising a second hydrophobic peptide segment consisting of 5 to 20 substituted or unsubstituted amino acids selected from the group consisting of L-phenylalanine, L-tryptophan, L-dopa, L-tyrosine, L-cysteine, S-benzyl-L-cysteine, and S-methyl cysteine.

5. The block copolypeptide of claim 4, wherein the first positively charged peptide segment is consisting of substituted or unsubstituted L-lysine or L-homoarginine.

6. The block copolypeptide of claim 4, wherein the second hydrophobic peptide segment is consisting of unsubstituted L-cysteine or S-benzyl-L-cysteine.

7. The block copolypeptide of claim 4, wherein the first positively charged peptide segment is selected from the group consisting of L-lysine₅, L-lysine₉, L-lysine₁₀, L-lysine₁₅, L-homoarginine₅, L-homoarginine₉, L-homoarginine₁₀, and L-homoarginine₁₅.

8. The block copolypeptide of claim 4, wherein the second hydrophobic peptide segment is selected from the group consisting of S-benzyl-L-cysteine₅, S-benzyl-L-cysteine₉, S-benzyl-L-cysteine₁₀, S-benzyl-L-cysteine₁₅, L-cysteine₅, L-cysteine₉, L-cysteine₁₀, and L-cysteine₁₅.

9. The block copolypeptide of claim 4, wherein at least one of the amino acids is is grafted with at least one selected from a group consisting of p-methoxybenzaldehyde, vanillin, cinnamaldehyde, catechol, indole, phenol, and phenyl.

10. The block copolypeptide of claim 4, having 3 to 24 multi-arms.

11. The therapeutic block copolypeptide of claim 10, wherein the first positively charged peptide segment is present in at least one arm.

12. A method for treating cancer, comprising:
administering an effective amount of the block copolypeptide of claim 1 to a subject in need thereof.

13. The method of claim 12, wherein the effective amount is from 5 mg/kg to 10 mg/kg bodyweight of the subject.

14. The method of claim 12, wherein the cancer is selected from group consisting of lung cancer, breast cancer, ovarian cancer, brain cancer, kidney cancer, oral cancer, esophageal stomach cancer, colon cancer, liver cancer, pancreas cancer, uterine cancer, endometrial cancer, cervical cancer, gastric cancer, skin cancer, testicular cancer, prostate cancer, and thyroid cancer.

15. The method of claim 14, wherein the cancer is a lung cancer.

16. The method of claim 12, wherein the block copolypeptide suppresses migration and adhesion of the cancer cells and/or inhibits expression of E-cadherin in the cancer cells.

17. The method of claim 12, wherein the block copolypeptide is a vector for carrying an anti-cancer agent.

18. The method of claim 17, wherein the anti-cancer agent is cisplatin, oxaliplatin, nedaplatin, lobaplatin, p53 gene, or a combination thereof.

19. A method for increasing the proportion of Akkermansia muciniphila (Akk) in the gut of a subject in need thereof, comprising:
administering an effective amount of the block copolypeptide of claim 1 to the subject.

20. The method of claim 19, wherein the block copolypeptide is administered intravenously, intra-arterially, intra-peritoneally, intramuscularly, intradermally, intratumorally, orally, dermally, nasally, buccally, rectally, vaginally, by inhalation, or by topical to the subject.
